(19) United States
(12) Patent Application Publication
(10) Pub. No.: US 2003/0049253 A1
(43) Pub. Date: Mar. 13, 2003

(54) Polymeric conjugates for delivery of MHC-recognized epitopes via peptide vaccines

(76) Inventors: Frank Q. Li, Montgomery Village, MD (US); Yong-Liang Chu, Rockville, MD (US); Jian-Tai Qiu, Rockville, MD (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 10/062,710

(22) Filed: Feb. 5, 2002

Related U.S. Application Data
(60) Provisional application No. 60/310,498, filed on Aug. 8, 2001.

(51) Int. Cl. A61K 39/395

(52) U.S. Cl. 424/144.1; 424/178.1

(57) ABSTRACT

A method and compositions for modulating an immune system response to an antigen in a mammal are disclosed. The method comprises administering to the mammal a conjugate comprising substantially particle-free hyaluronic acid (HA), or a polymer analogue thereof, covalently linked to a peptide that comprises a T cell epitope, or a plurality of epitopes. Typically, the epitope is defined by a sequence of at least about eight amino acids of the antigen.

This application claims priority from U.S. Provisional Application Serial No. 60/310,498 filed Aug. 8, 2001. The entirety of that provisional application is incorporated herein by reference

### BACKGROUND OF THE INVENTION

1. Field of the Invention

The present invention relates to methods and compositions for modulation of an immune response to an antigen by modulation of presentation to T cells of epitopes of that immunogen by Major Histocompatability Complex (MHC) molecules. More particularly, the invention provides immunogenic conjugates of polymers, such as hyaluronic acid and analogues thereof, and peptides or other molecules comprising epitopes recognized by MHC molecules, which conjugates are useful for modulating, that is, enhancing or diminishing, an immune system response to such epitopes.

2. Background of the Technology

Vertebrates have developed a sophisticated system to protect themselves against a wide variety of hazards including various viruses and microorganisms, such as bacteria and fungi, as well as genetic diseases, neoplasia, and effects of a variety of toxins. The system has evolved based on the ability to recognize self as distinct from non-self or “foreign”. A broad panoply of defense mechanisms are involved, including phagocytosis, lysis, such as complement mediated or perform mediated lysis, and killer r cells, such as cytotoxic T lymphocytes, natural killer cells, antibody dependent cytotoxic cells, and the like. Various cell types offer different mechanisms whereby the invader or endogenous diseased cell may be eliminated.

A key to the immune defensive mechanism is the T cell. For instance, it is well known that the adaptive immune system shows a much stronger response on second, as compared to first, encounter with an antigen. This phenomenon is exploited in vaccination, which works by inducing a state of lasting immunity known as immunological memory. Immunological memory requires activation of T lymphocytes specific for the vaccine antigen.

T cells have been found to be “restricted” in that they respond to an antigen in relation to one or a few specific transplantation antigens (now called major histocompatibility antigens, “MHC”) associated with their natural host. In vitro, T cells from a host of one haplotype respond to an antigen in relation to a transplantation antigen of a different haplotype host. The T cell receptor recognition repertoire appears to be narrower than the recognition repertoire of immunoglobulins produced by B cells. In addition, rather than directly binding to an antigen as do antibodies and other immunoglobulins, the T cell receptor appears to require concomitant binding to a foreign antigen and an MHC molecule.

MHC molecules are divided into two classes, Class I and Class II, where the former class is relatively ubiquitous on vertebrate cells, while the latter is relatively limited to lymphocytes, macrophages, and dendritic cells. Functionally different T cells appear to be activated in relation to one or the other class of MHC molecules. In the main, the nature of the activity of a T cell will vary with the Class of the MHC molecule to which it is complementary. In effect it appears that a T cell clone recognizes a specific antigen in conjunction with a specific MHC allele. Furthermore, variation in the antigen structure affects the nature of the response when the T cell, antigen, and antigen presenting cell are brought together. Depending upon the nature of the structural change, all three possibilities are encountered, namely, no change, increased stimulation or decreased stimulation of an immune response to the antigen.

More particularly, T lymphocytes detect foreign polypeptide antigens by recognizing—via the T cell receptor (“TCR”)—peptide fragments derived from the antigen. Most T lymphocytes, however, are MHC restricted, that is, they recognize only complexes of peptides bound to the highly polymorphic membrane proteins encoded by Class I and Class II MHC genes and presented (displayed) on the surface of an accessory cell (designated an antigen-presenting cell or “A.C.”), in which the antigen has been processed.

Antigens can be processed by one of two pathways, depending on their origin, inside or outside the A.C. In a first pathway, foreign material from outside a cell is engulfed by a specialized A.C. (often a macrophage or B-cell), which breaks down the material and complexes the processed antigen with Class II MHC molecules. In particular, MHC Class II molecules are synthesized in the endoplasmic reticulum with their antigenic peptide binding sites blocked by the invariant chain protein (Ii). These MHC Class II-Ii protein complexes are transported from the endoplasmic reticulum to a post-Golgi compartment where Ii is released by proteolysis and a specific antigenic peptide becomes bound to the MHC Class II molecule.

Class II MHC molecules are expressed primarily on cells involved in initiating and sustaining immune responses, such as T lymphocytes, B lymphocytes, macrophages, and the like. Complexes of Class II MHC molecules and immunogenic peptides are recognized by helper T lymphocytes (also known as helper/accessory T cells, “Th”) and induce proliferation of Th lymphocytes. Class II MHC complexes also stimulate secretion of cytokines by Th cells, resulting in amplification of the immune response to the particular immunogenic peptide that is displayed. In particular, Th1 cells produce interferon-γ and other cytokines that stimulate CTLs, while other cytokines produced by Th2 cells help B cells to produce antibodies.

A second antigen processing pathway is generally concerned with foreign proteins made within cells, such as virus-infected or malignant cells. These proteins are subjected to partial proteolysis within such cells so as to form peptide fragments that then associate with Class I MHC molecules and are transported to the cell surface for presentation to T cells. Class I MHC molecules are expressed on almost all nucleated cells, and complexes of Class I MHC molecules and bound immunogenic peptides are recognized by cytotoxic T lymphocytes (“CTLs”; also known as cytotoxic/suppressor T cells, “Tc/s”), which then destroy the antigen-bearing cells. CTLs are particularly important in tumor rejection and in fighting viral infections.

For a CTL to recognize an antigen in the form of a peptide fragment bound to the MHC class I molecule, that antigen must normally be endogenously synthesized by the cell and a portion degraded into small peptide fragments in the cytoplasm. Some of these small peptides translocate into a pre-Golgi compartment and interact with Class I heavy chains to facilitate proper folding and association with the subunit β2 microglobulin. The peptide-MHC Class I complex is then routed to the cell surface for expression and potential recognition by specific CTLs.

By these dual antigen processing pathways, appropriate defenses are generated against both exogenous and internally produced antigens. Thus, antigens taken up from the surroundings eventually elicit B cells to produce antibodies that protect the organism against a subsequent challenge by an agent comprising the exogenous antigen. On the other hand, antigens comprised of abnormal structures made within an abnormal or errant cell (for example a virus-infected or malignant cell) activate an immune response that eventually leads to killing of the errant cell. There is considerable interest in methods for better stimulating immune responses to antigens that are processed by either of these two pathways and presented by either MHC Class I or Class II molecules.

Class I transplantation antigens, however, are also involved in rejection of organ transplants. The immune system is able to recognize the foreign nature of the transplantation antigens present on the transplanted organ and attack the organ. In this situation, rather than protect the host from the foreign cells, the desired goal of immune system modulation is to diminish the subset of the immune system which is specific for attacking the organ transplant.

Much is known about the molecular basis of MHC recognition of antigens. Thus, MHC molecules contain at least two binding sites, namely, an antigen binding site, known as an “agretope” binding site, which is highly variable among MHC molecules, and a site which binds to the T cell, namely, the “T cell specific binding ligand”, which is highly conserved (see Bjorkman, P. J. et al., Nature 329:506-508 (1987) and Bjorkman, P. J. et al., Curr. Opin. Struct. Biol. 4:852-856 (1994)). Binding of MHC molecules on antigen presenting cells to the surface of T cells is in part mediated through agretope binding, that is, binding of an antigen to an MHC molecule acts as a signal to the MHC molecule to bind to the surface of a T cell (see Bjorkman, P. J. et al., 1994, supra). In addition, if the highly conserved MHC molecule recognition sites on the APC and the T cell are not of the same genetic composition, the APC cells will be recognized as “not-self” and the desired interaction cannot successfully occur.

The conserved T cell specific binding ligand of an MHC molecule binds to an adherence receptor molecule on an appropriate T cell. Thus, depending on the specificity of an adherence receptor molecule, T lymphocytes can be classified as CD4⁺ or CD8⁺. The CD4 adherence receptor recognizes MHC Class II molecules, while the CD8 receptor binds Class I. In addition, MHC restriction is further dependent on direct binding of the MHC molecule to certain parts of the TCR. CD4⁺ T cells that are activated by peptide-containing MHC Class II complexes serve as helper T cells, activating macrophages and antibody-producing B cells. In contrast, CD8⁺ T cells activated by Class I complexes comprising an immunogenic peptide become cytolytic T cells, thereby giving rise to cell mediated immunity to the disease causing agent comprising the peptide, for instance, a virus, without any humoral immunity (i.e., antibody) involvement.

T cells are therefore activated by the combination of (1) binding to the T cell surface of fragmented antigenic determinants bound to MHC molecules on the APC surface, and (2) binding to the T cell surface of the highly conserved region of the MHC molecule. Usually, binding by the fragmented antigenic determinants or the highly conserved region of the MHC molecules alone to the surface of the T cells does not give rise to activation of T cells since to do so would give rise to an unregulated and indiscriminate polyclonal activation of most, if not all, T cells and could result in pathogenic conditions.

Each fragmented antigenic determinant which binds to an MHC molecule and thereby stimulates T cells comprises at least two functionally distinct but potentially physically related amino acid sequences, namely, an “agretope” and an “epitope”. As noted above, an amino acid sequence that binds to an MHC molecule is referred to as an “agretope”. The agretope is a single unit of recognition that specifically binds to one or a limited number of MHC molecules. The agretope is defined by a set of agretopic amino acid residues, with the individual amino acid residues frequently separated.

The “epitope” is considered to be the basic element or smallest unit of recognition by a receptor, particularly immunoglobulins and T-cell receptors. An epitope may be comprised of portions of a polypeptide that are contiguous or noncontiguous in the amino acid sequence of the polypeptide, and the amino acids essential to the receptor recognition in each portion may be contiguous and/or non-contiguous in the sequence. Amino acids interspersed between agretopic residues of a peptide fragment of an antigen may provide epitopic recognition by themselves or in conjunction with one or more agretopic residues. Further, an amino acid sequence may have one or more different units of recognition, so that in referring to an agretope in a peptide, it should be understood that there may be a plurality of sets of agretopic amino acid units, where even the same set may vary in its binding affinity in different peptides, depending upon adjacent amino acids.

In view of the above knowledge, there has been substantial interest in using short peptides to affect an immune response in vivo and in vitro, to provide stimulation or inactivation of a particular response. Thus, appropriate immunogenic peptides might modulate a natural immune response to a particular event, either by activating particular lymphocytes to enhance a protective response or by deactivating particular lymphocytes to diminish or prevent an undesirable response.

Short synthetic peptides containing empirically identified immunogenic sequences have been used to present agretopes and epitopes of polypeptides to stimulate various vertebrate immune responses against diverse antigens. For example, U.S. Pat. No. 5,019,384 to Gefter et al. discloses methods and compositions for modulating the immune system, so as to selectively stimulate or inactivate lymphocytes in relation to a particular transplantation antigen (MHC molecule) content. The disclosed polypeptide reagents have one or two regions. A first region comprises oligopeptides having an amino acid sequence which provides for enhanced binding to target MHC molecule(s), that is, an agretope. The interspersed amino acids are referred to as the “epitope” (which includes epitopes, that is, the amino acids may define a plurality of recognition sites).

U.S. Pat. No. 5,196,512 to Bianchi et al. discloses synthetic peptides useful as universal carriers for the preparation of immunogenic conjugates and their use in the development of synthetic vaccines. In particular, this patent teaches that the synthetic peptide TT3, the amino acid sequence of which corresponds to the region 947-967 of the tetanus toxin, is recognized by different human T helper (Th) cell clones in association with a wide range of MHC alleles. The patent further discloses that the TT3 peptide and the peptide corresponding to the 947-960 epitope can be used as universal carriers in the preparation of immunogenic conjugates consisting of at least one of these peptides and a natural or synthetic hapten derived from a pathogenic agent of interest.

U.S. Pat. No. 5,919,639 to Humphreys et al. discloses a class of compounds referred to as effector compounds said to be useful in connection with the modulation of an immune response. Modulation refers to the ability of the effector compounds of the present invention to either enhance (antigen supercharging) or inhibit (immunosuppressant activities) antigen presentation, depending upon the nature of the particular effector compound and the therapeutic context. Disclosed effector compounds include peptides, modified peptides and peptidomimetics of a particular mammalian Ii key peptide. The patent discloses methods for enhancing presentation of an MHC Class II restricted antigenic peptide to a T cell, including contacting the following components under physiological conditions: an MHC Class II expressing antigen presenting cell; the mammalian Ii key peptide; the MHC Class II restricted antigenic peptide which, when added to the incubation mixture, is not in association with an antigen presenting cell; and a T cell which is responsive to the MHC Class II restricted antigenic peptide.

U.S. Pat. No. 5,993,819 to Haynes et al. discloses immunogenic preparations of peptides comprising amino acid sequences corresponding to antigenic determinants of the envelope glycoprotein of HIV, covalently coupled, directly or through a spacer molecule, to carrier molecules suitable for vaccination of mammals. Carrier molecules to which peptides of the invention are covalently linked (conjugated) are advantageously, non-toxic, pharmaceutically acceptable and of a size sufficient to produce an immune response in mammals.

Recently, there has been considerable progress in the analysis of structural requirements for immunogenic peptides bound to MHC Class I and II molecules. For reviews, see e.g., Janewa, C., Nature 353:3:852-8555 (1991); Rotzschke, O. and Falk K., Immunol Today 12:447-455(1991); Stauss. H. J., Immunol Today 20:180-183(1991); Tsomides, T. J. and Eisen H. N., Proc Natl Acad Sci USA 91:3487-3489 (1991). Thus it has been found that MHC Class I molecules bind short peptides of only about 8-12 amino acids and that MHC Class II molecules bind peptides of about 10-17 amino acids. Exceptions to these guidelines include formylated peptides, which are capable of MHC presentation with only about 5-7 amino acids (Kurlander et al., J. Immunol Dec. 15, 1999:163(12):6741-7; Seaman et al., Immunol May 1, 1999: 162(9):5429-36). Further, it has been found that peptide fragments resulting from processing of antigens are transported to the cell surface bound in a groove on the extracellular part of an MHC molecule.

For MHC Class I molecules, it has been found that individual amino acid side-chains, located at precise positions along the peptide, bind into the peptide-binding groove (Madden, D. R. et al., Nature 353:321-325 (1991)). The position of these binding pockets and the amino acids that line them can be different for different allelic MHC variants. Consequently, different MHC Class I molecules bind different sets of peptides, and allele-specific motifs have been found for various MHC alleles (Van Bleek, G. M. and Nathenson, H. G., Proc Natl Acad Sci USA 88:11032-11036 (1991); Falk K. et al., Nature 351:290-296 (1991); Jardetzky, T. S. et al., Nature 353:326-329 (1991)). For example, peptides binding HLA-A2.1 preferably have L or M in position 2 and V or L in position 9, i.e., in the C-terminal position (Rotzschke and Falk, 1991, supra).

As opposed to Class I molecules, Class II have a binding groove that is open and thereby able to accommodate longer peptides of 13-18 amino acids Nelson, C. A. et al., Rev Immunogenet. 1:47-59 (1999)). The majority of identified Class II-associated peptides have been of this length, although a crystallization model (Reinherz) suggests that a central core of as few as nine amino acids actually come into contact with the T cell receptor. Confirmation of this report is provided by recent published data that identify HIV-1 specific T helper epitopes of nine amino acids in length (Wilson C. C. et al., J. Virol. 75:4195-4207 (2001)).

Such detailed knowledge of structural requirements for both Class I and Class II MHC binding of peptides has also been applied to synthetic peptide vaccines. For instance, U.S. Pat. No. 6,037,135 to Kubo et al. discloses methods for determination of allele-specific peptide binding motifs for allele subtypes of human Class I MHC molecules (also referred to as human leukocyte antigens, “HLAs”). Immunogenic peptides comprising an MHC-restricted T cell epitope consisting of about 8-11 amino acid residues, which bind to an MHC molecule and induce an MHC-restricted cytotoxic T cell response, are identified by empirical testing of peptides comprising an MHC-binding motif for induction of a cytotoxic T cell response. Optimally the immunogenic peptide has a length of 9 or 10 amino acid residues, commensurate in size with endogenously processed viral peptides or tumor cell peptides that are bound to MHC Class I molecules on the cell surface. The peptides are said to be useful in vaccine compositions for administration to mammals, particularly humans, to treat and/or prevent viral infections and cancer.

U.S. Pat. No. 6,033,669 to Jondal discloses immunogenic conjugates useful for generating T cell immunity against tumor-associated carbohydrate structures or against carbohydrate structures expressed on infectious agents and/or infected host cells. The disclosed immunogenic conjugate comprises,(i) a peptide component capable of binding a MHC class I molecule;-and (ii) a carbohydrate component having the immunogenic specificity of the carbohydrate structure. The carbohydrate can be coupled to the carrier peptide aminoterminally, carboxyterminally, or it may be coupled to an internal amino acid.

U.S. Pat. No. 6,096,315 to Zimmerman et al. discloses heterofunctional cellular immunological reagents comprising at least two T cell specific binding ligands covalently linked together, wherein one of the T cell specific binding ligands binds to a specific class or subclass of T cells and another of the T cell specific binding ligands is an antigen associated with disease or a causative agent of disease, or epitope thereof. The particular T cell specific binding ligand which binds to a specific class or subclass of T cells can be selected so as to bind to all mature T cells, only mature cytotoxic T cells, helper T cells, suppressor T cells or a specific class or subclass thereof.

As another recent example of peptide immunogens, Peter, K. et al., “Induction of a cytotoxic T-cell response to HIV-1 proteins with short synthetic peptides and human compatible adjuvants,” Vaccine 19:4121-4129 (2001), discloses induction of a CTL response against multiple CTL epitopes present in HIV proteins using short synthetic peptides. Four HLA-A2.1 restricted peptides (RT 476-484, p17 77-85, gp41 814-823, RT 956-964) that showed stable binding to the HLA-A2.1 molecule in an in vitro binding assay were able to elicit a strong specific immune response in HLA-A2.1 transgenic mice when injected with a peptide (“P30”) used as a universal T cell helper epitope, in incomplete Freund adjuvant (IFA) or a nonionic emulsifier (Montanide® ISA 720). The use of biodegradable poly-L-glutamic acid (PLGA) microspheres (MS) as adjuvant was also successfully tested for all peptides.

Development of vaccine adjuvants for use in humans is reviewed in Singh, M. et al., “Advances in vaccine adjuvants”, Nat. Biotechnol. 17:1075-1081 (1999), which discloses that, currently, aluminum salts and the MF59 microemulsion are the only vaccine adjuvants approved for human use.

Hyaluronic acid (HA; also known as “hyaluronan”) is a ubiquitous extracellular matrix (ECM) component, present at high concentrations in the skin, where it is synthesized primarily by dermal fibroblasts and by epidermal keratinocytes. See, e.g., Terneer, C. C. et al., “Oligosaccharides of hyaluronan are potent activators of dendritic cells.” J Immunol. 165:1863-1870 (2000). In normal skin, HA exists as a high molecular weight (600,000-1,000,000 daltons) (HMW-HA) nonsulfated glycosaminoglycan (GAG) composed of repeating units of D-glucuronic acid-N-acetyl-D-glucosamine. Functional properties of HMW-HA are the maintenance and hydration of the cutaneous ECM, as well as the binding of various growth factors and smaller GAGs with specificity for cellular receptors, termed hyaladherins.

The physiological degradation of HMW-HA within the skin includes the uptake into keratinocytes, which is related to the high affinity HA receptor CD44 and intracellular fragmentation to intermediate sized fragments (INT-HA 60,000-300,000 daltons). Id. Fragmented HA is released by keratinocytes, passes the basement membrane, and is liberated without significant further catabolism by dermal cells into lymphatic vessels. These fragments are degraded within skin-draining lymph nodes. Alternatively, uptake and catabolism of HA from the blood stream by liver endothelial cells have been described. Finally, during inflammation, platelet-derived chemotactic factors like fibrin stimulate the influx and activation of fibroblasts. These cells directly degrade the surrounding ECM components by the secretion of hyaluronidase resulting in increased tissue concentrations of small HA fragments (sHA). Furthermore, cleavage of HA can be induced by reactive oxygen species released for example by granulocytes or in UV-irradiated skin, demonstrating that different proinflammatory stimuli can trigger unspecific degradation of HA.

Various forms of HA and other similar polymers have been described for use in delivery of foreign substances such as drugs. For instance, U.S. Pat. No. 4,636,524 to Balazs et al. discloses cross-linked gels of HA, alone or mixed with other hydrophilic polymers, containing various substances or covalently bonded to low molecular weight substances, and processes for preparing them. The products are said to be useful in numerous applications including cosmetic formulations and as drug delivery systems. One example illustrates slow release of a low molecular weight substance (hydroxytryptamine binoxolate) dispersed in a matrix of cross-linked HA. U.S. Pat. No. 5,166,331 to delta Valle et al., U.S. Patent No. 5,688,931 to Nogusa et al. All describe HA preparations of various molecular weights for use in administration of biologically active components to animals and humans.

U.S. Pat. No. 5,856,299 to Roughed et al. discloses active esters of carboxyl polysaccharide and semisynthetic derivatives of carboxyl polysaccharide, wherein all or part of the carboxyl groups thereof are esterified with an aromatic alcohol, a substituted aromatic alcohol, an aromatic heterocyclic alcohol, a substituted aromatic heterocyclic alcohol, an N-hydroxylamine, or a combination thereof. These active esters can be used for the preparation of modified carboxyl polysaccharide or modified semisynthetic derivatives of such carboxyl polysaccharide, in the form of esters, thioesters, or amides. Various esters of HA containing different peptides of 3-6 amino acids are exemplified, in either soluble or microparticulate forms. The modified polysaccharides produced from active esters of the invention are said to be useful in the preparation of health care and surgical articles for internal or external use, such as microcapsules, microspheres, threads, films, gauzes, sponges, etc., to be used, for example, in wound care, tissue healing and repair, prevention of tissue adhesion, and controlled-release systems for biologically active substances such as amino acids, peptides, and proteins.

A more recent example of drug delivery compositions comprising HA and like polymers is provided by U.S. Pat. No. 6,063,370 to Dadey, which discloses macromolecular drug complexes containing a drug such as insulin, and a polymer having a plurality of acid moieties such as carboxyl moieties or phosphonic acid moieties. Compositions containing the macromolecular complexes are administered to individuals suffering from a disease, and the complexes release the drug, in vivo, to treat the disease, and to reduce, eliminate, or reverse complications associated with the disease. In particular, the patent describes polymers having a plurality of acid moieties selected from the group consisting of carboxyl, phosphate, phosphonate, sulfate, sulfonate, phenolic, and mixtures thereof, for noncovalent complexing with the quaternary ammonium nitrogen atom of the drug. The drug may be a polypeptide or a protein, among others, and the polymer may be HA, among others.

U.S. Pat. No. 6,180,601 to Jederstrom discloses a freeze-dried soft, flexible and continuous matrix of low-molecular weight HA or salt thereof, in which the molecular weight of the HA is preferably between 50,000 and 200,000 Da, containing at least one peptide or protein. It also discloses a pharmaceutical composition in the form of a layer which is characterized by this freeze-dried low-molecular weight HA containing at least one peptide or protein. The drug is preferably chosen from at least one of GH, IGF-I, IGF-II and/or EGF and could be mixed with an antibiotic agent. The patent further discloses a method for accurately obtaining a predetermined dosage of a topically administerable drug which is characterized by freeze-drying a water solution of low-molecular weight HA and the peptide or protein to form a layer.

U.S. Pat. No. 6,291,671 BI to Inoue et al. discloses a method for preparing a drug complex in which a polysaccharide derivative having carboxyl groups and a residue of a drug compound are bound to each other by means of a spacer comprising an amino acid or a spacer comprising 2 to 8 peptide-bonded amino acids, or a drug complex in which a polysaccharide derivative having carboxyl groups and a residue of a drug compound are bound to each other without the spacer, characterized in that an organic amine salt of the polysaccharide derivative having carboxyl groups is reacted with the drug compound or the spacer bound to the drug compound in a non-aqueous system.

Interactions between HA or similar polymers and the immune system also have been studied for many years. For instance, T cell-independent B cell triggering has been studied using various hapten-coupled polymeric “carriers” with dinitrophenol (DNP) as a model hapten. See, e.g., Klaus G. G. et al., “The immunological properties of haptens coupled to thymus-independent carrier molecules. III. The role of the immunogenicity and mitogenicity of the carrier in the induction of primary IgM anti-hapten responses”, Eur. J. Immunol. 5:105-111 (1975). According to these authors, “[w]ith these antigens, the carrier per se elicits T cell-independent IgM antibody formation, and the hapten-conjugates elicit T cell-independent IgM anti-DNP responses.” More particularly, the authors disclose that conjugates of the hapten (2,4-dinitrophenol-lysine; DNP-lys) and two putatively nonimmunogenic polymers, HA and poly-y-D-glutamic acid, induce significant primary IgM anti-DNP responses in C3H mice. Preparations of various immunogenic (Type 3 pneumococcal polysaccharide (Sill), levan, E. coli lipopolysaccharide) and nonimmunogenic (HA and poly-glutamic acid) polymers were tested for their ability to act as polyclonal mitogens in vitro. Purified HA was substituted with DNP-lysine by cyanogen bromide activation of the polymer. There was no apparent correlation between the capacity of various polymers to induce lymphocyte proliferation and their “potency” as carriers for the generation of a primary IgM anti-DNP response. Furthermore, while low doses of lipopolysaccharide elicited “polyclonal” antibody formation in vivo, high doses of SIII, levan and HA did not. These results were said to indicate that T cell-independent B cell triggering is dependent on the polymeric nature of the antigen, and that polymers need not be immunogenic or mitogenic to act as carriers for the induction of primary IgM anti-hapten antibody responses.

Eur. J. Immunol. 11:212-20 (1981), indicates that DNP conjugates of Ficoll, hydroxyethyl starch, levan, dextran, type 3 pneumococcal capsular polysaccharide SIII, pectin, alginic acid and HA at various epitope densities were tested for their capacity to inhibit secondary IgG anti-DNP antibody responses using an in vivo transfer system. Conjugates of the acidic polysaccharides were markedly more effective than those of uncharged polysaccharides. Conjugates with high epitope densities were particularly tolerogenic.

U.S. Pat. No. 4,725,585 to Wenge et al. discloses a method of enhancing or regulating the host defense of a mammal, said method comprising administering to a mammal a therapeutically effective amount of HA. One exemplified embodiment is a method for normalizing a reduced phagocytic activity exerted by the granulocytes of a mammal, comprising administering subcutaneously or intramuscularly to the mammal a non-antigenic HA preparation containing HA or a physiologically acceptable salt thereof in a therapeutically effective amount. One type of HA said to be suitable for the discloses purposes is the essentially pure HA disclosed in U.S. Pat. No. 4,141,973. This HA has an average molecular weight of at least about 750,000 and a protein content of less than 0.5% by weight.

More recently, effects of HA on antigen presenting cells (APCs) have been investigated. For instance, Termeer, C. C. et al., noted that HA in the extracellular matrix exists physiologically as a high molecular weight polymer but is cleaved at sites of inflammation, where it is contacted by DC. To determine the effects of HA on DC, HA fragments of different size were established. Only small HA fragments of tetra- and hexasaccharide size (sHA), but not of intermediate size (m.w. 80,000-200,000) or high m.w. HA (m.w. 600,000-1,000,000) induced immunophenotypic maturation of human monocyte-derived DC. Likewise, only sHA increased DC production of the cytokines IL-1β, TNF-α, and IL-12 as well as their allostimulatory capacity. These effects were highly specific for sHA, because they were not induced by other glycosaminoglycans such as chondroitin sulfate or heparan sulfate or their fragmentation products. sHA-induced DC maturation was shown not to involve the HA receptors CD44 or the receptor for HA-mediated motility, because DC from CD44-deficient mice and wild-type mice both responded similarly to sHA stimulation, whereas the receptor for HA-mediated motility is not detectable in DC. According to the authors, these findings suggest that during inflammation, interaction of DC with small HA fragments induces DC maturation.

A recent review article by Camenisch T. D. and McDonald J. A., “Hyaluronan: is bigger better?” Am J Respir Cell Mol Biol. 23:431-433 (2000) discloses that various biological activities attributed to HA vary depending upon molecular mass and discusses possible mechanisms of such variability.

Diverse forms of HA and similar polymers, including cross-linked, insoluble and/or particulate forms, also have been used to prepare immunostimulatory compositions comprising a peptide antigen. For instance, U.S. Pat. No. 5,008,116 to Cahn discloses immunostimulatory microspheres, for instance, a macroporous microsphere comprising a particle and an antigenic component, selected from the group consisting of whole inactivated pathogens and subunits thereof, cDNA derived polypeptides, recombinant viruses, synthesized polypeptides and anti-idiotypes, and whole microorganisms or animal cells, wherein the antigenic component is physically entrapped in or chemically crosslinked to the interior of the particle, and the particle is comprised of a polymer. The particle is sufficiently small that it can be injected with a syringe, e.g., 1 to 100 μm preferably below about 50 μm diameter. Suitable polymers for making the particle are said to include crosslinked gels of HA disclosed in U.S. Pat. No. 4,636,524.

U.S. Pat. No. 5,665,383 to Grinstaff et al. discloses methods for the preparation of immunostimulating agents, such as a vaccine, for in vivo delivery, in which a biologic is associated with a polymeric shell formulated from a biocompatible material. The size range of particles obtained by the invention method is between 0.1 micron (μm) to 20 microns, with a preferred size range said to be 0.5 to 10 microns and the most preferred range, 1 to 5 microns. The patent states that essentially any material, natural or synthetic, bearing sulfhydryl groups or disulfide bonds within its structure may be utilized for the preparation of a disulfide crosslinked shell. The sulfhydryl groups or disulfide linkages may be preexisting within the structure of the biocompatible material, or they may be introduced by a suitable chemical modification. For example, naturally occurring biocompatible materials such as proteins, polypeptides, oligopeptides, polynucleotides, polysaccharides (e.g., starch, cellulose, dextrans, alginates, chitosan, pectin, HA, and the like), lipids, and so on, are said to be candidates for such modification. The patent further states that the structure of the polymeric shells allows for the delivery of antigenic material such as proteins, peptides, nucleic acids, polysaccharides, etc., by injection and other routes, and that the antigenic material may be contained within, or form part of, the polymeric shell.

U.S. Pat. No. 5,674,495 to Bowersock et al. discloses a vaccine composition including an antigen dispersed in an alginate gel. The alginate gel is preferably in the form of discrete particles coated with a polymer. The patent says that vaccination of vertebrate species can be accomplished by administering the alginate-based vaccine compositions orally, and further states that similar carbohydrate polymers could be used to achieve the same function of encapsulation of any antigen including live viruses or bacteria or parasites (protozoa or helminths, strongyles, etc.) in polymer particles, including but are not limited to gelatin, dextran, HA, and starch.

U.S. Pat. No. 6,150,461 to Takei et al. discloses a carrier for delivering a foreign substance to a target organ which comprises a graft copolymer in which HA is grafted onto a polymer comprising a main chain. The main chain has a part capable of binding to the foreign substance electrostatically, and contains one or more monomer unit having side chains with an amino or imino group capable of coupling to HA. The carrier is said to be useful for delivering a foreign substance to a target organ, especially liver. Among many foreign substances that are said to be deliverable by the carrier are peptides and various vaccines. The carrier is exemplified by a hyaluronate-grafted poly-(L-lysine) copolymer which is mixed with DNA for delivery to the liver. The patent says that the molecular weight of HA is not critical, with preparations having average molecular weight of less than 100,000, preferably from 1,000 to 50,000, being suitable in terms of reactivity with poly-amino acids (PAA) and the ability to recognize a target organ.

Although many short immunogenic peptides have been identified which could be useful in vaccines for modulating immune responses to various antigens, there remains a need for better delivery systems and adjuvants for preparing more practical and efficacious vaccines from such peptides. For instance, peptide delivery compositions requiring cross-linked or insoluble polymers, or particulate forms of such polymers, present major problems for manufacturing ease and 110 reproducibility, as well as various limitations on delivery routes and devices.

### SUMMARY OF THE INVENTION

The present invention meets the above-identified need by providing methods and compositions for modulating an immune system response to an antigen in a mammal, involving administering a conjugate comprising a particle-free polymeric hyaluronic acid analogue covalently linked to a peptide that comprises a T cell epitope of the antigen, where the T cell epitope is recognized by an MHC molecule of the mammal. Thus, as described herein below, the present inventors have discovered that covalent linking of hyaluronic acid to short synthetic peptides comprising a T cell epitope surprisingly increases T cell and B cell responses to the peptide, compared to responses obtained with peptide alone, even when the hyaluronic acid is used in a non-crosslinked, particle-free form. Increased immune system responses to peptides observed with conjugates of the invention include both cytotoxic T cell responses and antibody responses directed to the antigen. For instance, Example 2, below, surprisingly demonstrates that the humoral (antibody) immune response to a peptide derived from the V2 region of HIV-1 (called “peptide T”) in mice vaccinated with the peptide linked to HA was comparable to control mice with this peptide in complete Freund's adjuvant (CFA). Thus, the present invention unexpectedly provides for effective peptide vaccines without the use and disadvantages of adjuvants.

In contrast, the prior art appears to comprehend only that (1) T cell-independent B cell triggering can be obtained using various hapten-coupled polymeric “carriers” (e.g., dinitrophenol (DNP) as a model hapten linked to hyaluronic acid; see, e.g., Klaus, G. G. et al., supra), or (2) certain cross-linked, particulate forms of hyaluronic acid and related polymers may enhance immunogenicity of peptides or proteins that are either covalently or non-covalently associated with the particles (see, e.g., U.S. Pat. Nos. 5,008,116; 5,665,383; 5,674,495; and 6,150,461, cited hereinabove).

Accordingly, one aspect of the present invention relates to a method of modulating an immune system response to an antigen in a mammal in need of such modulation, comprising administering to that mammal a conjugate comprising a hyaluronic acid polymer analogue covalently linked to a peptide that comprises a T cell epitope of that antigen that is recognized by an MHC molecule of the mammal.

The hyaluronic acid polymer analogue of the invention conjugates includes any linear or branched glycosaminoglycan polymer comprising at least two, preferably at least ten, repeating disaccharide units, where the repeating disaccharide units include sugar acids naturally found in linear polymeric forms of hyaluronic acid (HA), amylose, dextran and chitosan or in branched HA, amylopectin and hemi-cellulose, as well as those in chondritic sulfate, dermatan sulfate, heparan sulfate, keratan sulfate, and derivatives thereof. Preferably, the hyaluronic acid polymer analogue is a polysaccharide containing a carboxyl group which, as defined herein below, includes a polysaccharide that naturally contains a carboxyl group in its structure, such as hyaluronic acid, pectic acid, alginic acid, chondroitin or heparin, as well as a polysaccharide naturally containing no carboxyl groups in which hydrogen atoms in a part or all of the hydroxyl groups are substituted by a carboxy C₁₋₄ alkyl group. More preferably, the hyaluronic acid polymer analogue of the invention conjugates is hyaluronic acid (HA), as defined below, including naturally occurring forms such as found in skin or other tissues of animals, including humans, typically a high molecular weight (e.g., 600,000-1,000,000 daltons) nonsulfated glycosaminoglycan (GAG) composed of repeating units of D-glucuronic acid (GicA) and N-acetyl-D-glucosamine (GlcNAc). HA preparations of various molecular weights may be used in the invention conjugates, including the aforementioned high molecular weight forms found in animal tissues, as well as the following: oligomers consisting of from at least about four to about six monomeric saccharides; polymers of intermediate molecule weights (e.g., about 50,000 to about 100,000 daltons); and polymers of higher molecular weights (e.g., about 500,000 to about 730,000 daltons). Particularly preferred are HA preparations having molecular weights in the range of 6,000,000-8,000,000 daltons, as described in the Examples, below.

Antigens from which epitopes may be selected for use in the present invention include, for instance, whole or partial foreign proteins derived from infectious organisms (e.g., viral, bacterial, fungal, or parasitic proteins), as well as native or mutant proteins expressed or overexpressed in a disease state (e.g., β-amyloid, cancer associated proteins). Non-protein antigens, such as polysaccharides or glycolipids, or part-protein antigens, such as glycoproteins, are also suitable for use in the present invention. An antigen suitable for use in the present invention includes at least one at least one T cell epitope capable of stimulating a cellular or humoral response. B cell epitope may also be included in compositions and methods of the present invention. Methods for identifying epitopes, including MHC-recognized T cell epitopes, particularly within protein antigens, are further detailed in the present specification and various disclosures referenced herein.

At least one peptide use in each invention conjugate comprises a T cell epitope that is recognized by an MHC molecule of the mammal to which the conjugates is administered. Preferably, the T cell epitope of the invention conjugate is one that is defined by a linear sequence of at least about eight amino acids of a desired antigen. As noted above, MHC Class I molecules are known to specifically bind (recognize) short peptides of only about 8-12 amino acids, and MHC Class II molecules bind peptides of about 9-18 amino acids. A single T cell epitope of the present conjugates typically comprises, therefore, a sequence of about 8 to about 18 amino acids from the antigen of origin.

Accordingly, a peptide comprising an MHC-recognized T cell epitope in a conjugate of the present invention typically comprises a sequence of about 8 to about 18 amino acids of a desired antigen. Longer peptides are included, however, which may comprise more than one T cell epitope of an antigen and/or other amino acid sequences from the same antigen (e.g., a B cell epitope or antibody binding site), or an amino acid sequence from a completely unrelated source. Of course, multiple peptides also may be used in a single conjugate, for instance, to combine multiple T cell epitopes from one or more antigens, or to combine a B cell epitope with one or more T cell epitopes from the same or a different antigen.

Further, besides peptides having an amino acid sequence identical to an epitope sequence in the desired antigen, in the present context peptides that comprise a T cell epitope of an antigen include modified (mutant) forms of such amino acid sequences having a molecular topology equivalent to that epitope sequence, produced, for instance, by recombinant means or chemical or enzymatic modification of a peptide. Also included in “peptides that comprise a T cell epitope” according to the present invention are non-peptide compounds that mimic peptides, having a molecular topology equivalent to the desired peptide epitope, which can be made by methods known in the art (see, e.g., U.S. Pat. No. 5,998,577 to Geysen which discloses a method of detecting or determining a sequence of monomer molecules that has molecular topology equivalent to that of a peptide epitope, where such non-peptide mimetics of epitopes, not necessarily containing any amino acid, are called “mimotopes”).

In another aspect, the invention provides a method of modulating an immune system response to an antigen using a conjugate as described above, where the T cell epitope is recognized by an MHC Class I molecule and by a CD8⁺ T cell of said mammal. Typically, such a T cell epitope is from an antigen of a pathological agent, such as a virus or a tumor cell, for which a cellular immune response is needed to protect the host from the pathological agent (prophylactic immunization), or to eliminate the agent from the host after the agent is established therein (therapeutic immunization).

In another aspect of the invention method, above, the T cell epitope is recognized by an MHC Class II molecule and by a CD4⁺ T cell of the mammal to which the conjugate is administered. Depending on the application, the epitope may be selected to be recognized by a Thl CD4⁺ T cell of the mammal, thereby to increase or enhance a cellular immune response (comprising a cytotoxic T lymphocyte that recognizes the antigen), or by a Th2 CD4⁺ T cell of the mammal, to enhance a humoral response (comprising an antibody that recognizes the antigen). In the former case, the epitope recognized by a Thl CD4⁺ T cell may advantageously be administered in combination with a T cell epitope recognized by a CD8⁺ T cell (CTL), while in the latter, the epitope recognized by a Th2 CD4⁺ T cell may advantageously be administered in combination with a B cell epitope.

Typically, the method of the invention will be used in a manner where the immune system response to an antigen after administration of the conjugate is increased, as in enhancing a prophylactic or therapeutic immune response to an antigen of a pathological agent or tumor cell. However, it is also desirable to use the invention in a manner to suppress or inhibit an immune response to an antigen, for instance, where the antigen is an antigen of a tissue or organ transplanted to a mammal or the subject of an autoimmune reaction in the mammal. Similarly, some antigens are known to block or inhibit immune responses through interactions with cellular receptors, such as the T cell receptor. Thus, the present invention also includes a method where the administration of the T cell epitope conjugate results in a decreased immune system response wherein the antigens used with the present invention function to inhibit or block signals of lymphocytes that recognize said antigen when presented with MCH Class II molecules. For example, Gefter et al., supra, discloses methods and peptide compositions for modulating the immune system so as to selectively inactivate lymphocytes in relation to a particular transplantation antigen (MHC molecule) content by using antigens that bind the T cell receptor but prevent signal transmission. Similarly, Humphreys et al., supra, discloses a class of compounds referred to as effector compounds said to be useful in connection with the modulation of an immune response, including either enhancement (antigen supercharging) or inhibition (immunosuppressant activities) of antigen presentation, depending upon the nature of the particular effector compound and the therapeutic context. Disclosed effector compounds include peptides, modified peptides and peptidomimetics of a particular mammalian Ii key peptide that serves as the internal ligand of a Class II MHC molecule.

While not being bound by any particular theory of how covalent linking of short peptides to polymeric hyaluronic acid of the present invention increases T cell responsiveness to the peptides, the inventors believe that the covalent conjugates of hyaluronic acid and peptides of the invention act by improving MHC presentation of a T cell epitope in the covalently linked peptide.

Accordingly, another of aspect of the present invention relates to a method of improving MHC presentation of a T cell epitope of an antigen in a mammal in need of same. This method comprises administering to the mammal a conjugate of the invention, comprising a substantially particle-free hyaluronic acid or polymer analogue covalently linked to a peptide that comprises a T cell epitope recognized by an MHC molecule of the mammal, where the T cell epitope is defined by a sequence of at least about eight amino acids of the antigen, as described herein.

In yet another aspect, the invention provides a pharmaceutical composition for administration to a mammal for improving MHC presentation of a T cell epitope of an antigen in that mammal. This composition comprises a conjugate comprising a substantially particle-free hyaluronic acid or polymer analogue thereof, covalently linked to a peptide that comprises a T cell epitope recognized by an MHC molecule of the mammal, where the T cell epitope is defined as above. Still another aspect of the invention is a composition of matter comprised of substantially particle-free hyaluronic acid, or a polymer analogue thereof, covalently linked to a peptide that comprises a T cell epitope recognized by an MHC molecule of a mammal.

Other features and advantages of the present invention will be readily appreciated as the same becomes better understood by reference to the following detailed description.

### BRIEF DESCRIPTION OF THE FIGURES

FIG. 1 shows quantification of cytotoxic lymphocyte (CTL) responses by intracellular staining and flow cytometry in splenocytes of mice immunized with a peptide derived from the p24 region of the HIV-1 Gag protein (the p7g peptide) covalently linked to HA (“HA-p7g”), as described in Example 1, demonstrating clonal expansion of CD4⁺ cells and a marked CTL response with CD8⁺ proliferation and INF-γ expression by CD8⁺ but not CD4⁺ cells. Negative control mice immunized with saline (“Naive”) demonstrated the expected distribution of CD4⁺ and CD8⁺ cell populations with no evidence of INF-γ expression. Mice immunized with free p7g peptide (“p7g”) showed a similar CD4/CD8 profile with no CTL or INF-γ expression.

FIG. 2 shows results of Enzyme-Linked Immunostaining Spot (“ELISpot”) assays indicating that p7g-specific CD8⁺ cells obtained from mice vaccinated with a p7g-HA conjugate did not express IFN-γ in the absence of p24 peptide stimulation, whereas when cells from the same animals were cultured in the presence of p24 peptide, IFN-γ secreting p7g-specific CD8⁺ cells were detected and ranged from 500 to 3200 SFU per million cells (FIG. 2A). This result represents a 10-50 times greater antigen specific CTL response than observed with free peptide (FIG. 2B). Mice were immunized as outlined in Example 1 via different routes and were challenged intraperitoneally (i.p.) with a recombinant vaccinia virus vector encoding HIV-1 Gag (10⁷ PFU per mouse) at 30 days after the peptide vaccination. Splenocytes were harvested 5 days after the recombinant vaccinia virus challenge and cultured in vitro with or without p7g peptide (aa AMQMLKETI) for 24 h. The spot numbers are the means of the triplicates. Error bars indicate the standard deviations from triplicated cultures.

FIG. 3 shows results of assays for gamma interferon (IFN-γ) in supematents of mouse spleen cells cultured with p7g peptide in parallel with the ELISpot assays of FIG. 2. Culture supernatants were collected 24 hours after addition of peptide, and the concentrations of were determined by enzyme-linked immunosorbent assay (ELISA).

FIG. 4 illustrates that the humoral immune response to an HIV-1 peptide (peptide T) in mice vaccinated with the peptide linked to HA was comparable to control mice with a peptide derived from the V2 region of HIV-1 (“peptide T”) in complete Freund's adjuvant (CFA). IgG titers were compared 56 days following the second vaccination with each peptide T vaccine formulation. Results were obtained in 1:100 dilutions.

FIG. 5 shows that antibody titer to a peptide representing human amyloid β amino acid residues 1-42 (Aβ42 peptide) in mice vaccinated with the peptide linked to HA was substantial whereas there was no detectable anti-peptide antibody in control mice immunized with Aβ42 peptide alone. Mice were immunized with 100 μg Aβ42 peptide per injection. Aβ42 in PBS alone was injected as control. Mice were primed s.c. on day 0 and boosted on day 14. Detection used goat anti-mouse immunoglobulin conjugated to alkaline phosphatase and p-nitrophenyl phosphate.

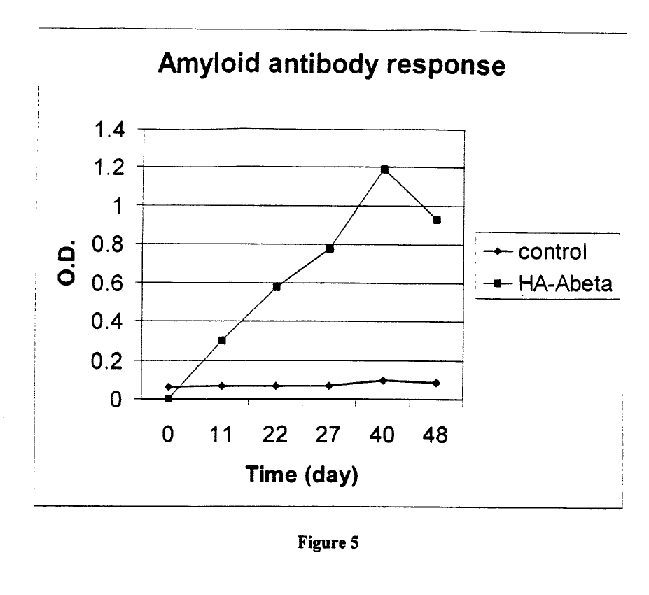

### DETAILED DESCRIPTION OF THE INVENTION

The potential advantages of peptide vaccines are appreciated, but have not been achieved by methods of the prior art. The present invention includes methods for making and using peptide vaccine compositions for preventing or treating disease in a mammal, preferably a human subject, which offer improved humoral and cell-mediated immune responses to a peptide consequent to covalent linkage of the peptide to a hyaluronic acid polymer or analogue thereof. The cell-mediated response to disease-associated antigens is known to play a critical role in preventing or eradicating, for example, infectious diseases and cancers. By improving the in vivo presentation of disease-associated antigens via covalent linkage to hyaluronic acid polymers and analogues thereof, the peptide vaccines of the present invention induce a significantly stronger cell-mediated immune response against the disease than free antigen or compositions with antigen simply mixed in HA polymer analogues. The present invention succeeds in inducing the desired specific and strong cell-mediated response in a mammal, which is central in preventing or treating the disease.

In contrast to the prior art, the compositions of the present invention utilize polymers, such as hyaluronic acid, not as generalized drug delivery agents or compounds intended to alter pharmacokinetic properties of a particular drug, but for improving immunogenicity of peptides recognized by Class I and/or II MHC molecules, thereby improving the cell-mediated response to the disease in the recipient. Furthermore, the vaccine compositions of the present invention do not require traditional adjuvants, such as alum, to induce an immune response, either a humoral (antibody) or a cell-mediated immune response, to an immunogen such as a disease-associated peptide or other antigen. Thus, peptide vaccine compositions of the present invention do not require additional adjuvants yet induce a stronger cell-mediated response than peptide vaccines of the prior art.

The present invention requires at least one covalent linkage between a peptide comprising an MHC-recognized epitope of an antigen and a soluble glycosaminoglycan polymer, wherein the linkage does not yield a microparticle or cross-linked matrix.

The present invention also provides for peptide vaccines comprising a plurality of peptides conjugated to HA polymer analogues. Multi-peptide HA polymer analogue compositions may be prepared according to the methods detailed below wherein at least two distinct peptides are separately conjugated to an HA polymer analogue and then combined, or alternatively, at least two distinct peptides are combined prior to the conjugation step.

Definitions

Because the present invention departs from the prior art pertaining to polymer carriers and peptide vaccine compositions, the following definitions are relied upon throughout the specification.

Hyaluronic Acid. As used herein, the term “hyaluronic acid” (HA; also known as hyaluronan) includes naturally occurring forms such as found in skin or other tissues of animals, including humans, typically a high molecular weight (600,000-1,000,000 daltons) nonsulfated glycosaminoglycan (GAG) composed of repeating units of D-glucuronic acid-N-acetyl-D-glucosamine.

Various forms of HA are known in the art and/or commercially available, derived from a variety of sources, and may be obtained as salts thereof. For example, sources of HA include human umbilical cord, rooster comb, streptococcus species, vitreous gel, and synthetic and recombinant preparations (Biochem. Biophys. Acta. 1380:377-388 (1998); U.S. Pat. No. 6,090,596). The following references teach additional sources and processes of the manufacture and recovery of HA polymer analogues: U.S. Pat. No. 4,141,973 teaches hyaluronic acid fractions having an average molecular weight greater than about 750,000; U.S. Pat. No. 4,141,973 teaches HA manufacture processes for obtaining average molecular weights of 50,000-100,000; 250,000 to 350,000; and 500,00-730,000; and U.S. Pat. No. 5,166,331 discloses an HA fraction with molecular weights between 50,000 and 100,000, and a second fraction having molecular weights between 500,000 and 730,000, and methods for preparing fractions of various molecular weight ranges by cleavage of high molecular weight HA and separation of fragments by ultrafiltration. Pharmaceutical grade preparations of HA approved by the FDA for human administration are commercially available; see, for instance, Example 1, below.

The molecular weight of HA fractions may be varied using methods well known in the art. For example, low molecular weight HA may be obtained from synthetic sources or the degradation of high molecular weight compositions by heat treatment (10 minutes at 100° C.), or enzymatic digestion with hyaluronidase and size exclusion chromatography (Camenisch and McDonald, Am. J Respir. Cell Mol Biol. 23:431-433 (2000)).

The hyaluronic acid used in the invention may further include a core glycan, such as Gal-Gal-Xyl repeats, and/or linkage to a protein core such as N- and O-linked glycoproteins found in naturally occurring proteoglycans. It is understood in the art that an HA polymer that includes a protein core will be depleted of such protein cores when administered in vivo.

Polysaccharide containing a carboxyl group. In the present invention, the term “polysaccharide containing a carboxyl group” means a polysaccharide which naturally contains a carboxyl group in its structure, such as hyaluronic acid, pectic acid, alginic acid, chondroitin or heparin. In addition, the term also means a polysaccharide naturally containing no carboxyl groups such as pullulan, dextran, mannan, chitin, inulin, levan, xylan, arabin, mannoglucan or chitosan, in which the hydrogen atoms in a part or all of the hydroxyl groups are substituted by a carboxy C₁₋₄alkyl group or in which at a part or all of the hydroxyl groups a polybasic acid is introduced through an ester linkage.

Hyaluronic Acid (HA) Polymer Analogue. The term “hyaluronic acid polymer analogue” as used herein includes hyaluronic acid, as defined herein, and also any linear or branched glycosaminoglycan polymer comprising at least two, preferably at least ten, repeating disaccharide units. The repeating disaccharide units include the sugar acids naturally found in linear polymeric forms of hyaluronic acid (HA), amylose, dextran and chitosan or in branched HA, amylopectin and hemi-cellulose, as well as those in chondritic sulfate, dermatan sulfate, heparan sulfate, keratan sulfate, and derivatives thereof.

A hyaluronic acid polymer analogue must be capable of forming at least one covalent bond between at least one disease associated peptide antigen and one disaccharide unit present in said HA polymer analogue, wherein the covalent linkage of a hyaluronic acid polymer to said peptide does not result in an insoluble complex or microparticle.

The basic chemical structure of the HA polymer analogues of the present invention comprise repeating dissacharide sugar acids with the formula (A-B)n, wherein n is at least 2 and preferably at least about 4 to about 16, more preferably at least about 4 to about 10. For instance, HA per se is comprised of alternating copoly(beta-glucuronic acid-[1→3]-beta-N-acetylglucosamine[1→4] or, according to the above formula wherein A is abbreviated as “glucuronic acid” (GIcA) and B is abbreviated as “N-acetylglucosamine” (GlcNAc), and n is at least 2, preferably at least about 4, more preferably at least about 10, and more preferably still at least about 16.

Irrespective of source, the basic chemical disaccharide structure of HA polymer analogues is functionally equivalent and in some instances (e.g., HA per se) identical to HA. Functionally equivalent HA polymer analogues may comprise the following sugar acid residue combinations: A=GlcA with B=GalNAc (chondroitin sulfate), A=GlcA or IdA (iduronic acid) and B=GalNAc (dermatan sulfate), A=GlcA or IdA and B=GlcNAc (heparan sulfate) and A=Gal and B=GIcNAc, where Gal represents a galactose, GlcN represents a glucosamine. HA polymer analogues of the invention also include the following synthetic polymers: polyphosphazines, poly(vinyl alcohols), polyamides, polycarbonates, polyvinyl ethers, polyvinyl esters, polyvinyl halides, polyvinylpyrrolidone, polyglycolides, polysiloxanes, polyurethanes, and synthetically modified natural polymers such as alkyl celluloses, hydroxyalkyl celluloses, cellulose ethers, cellulose esters, and nitrocelluloses.

HA polymer analogues of the present invention may be further derivatized to alter surface charge or functional groups for covalent linkage to a peptide. For example, the native form of HA lacks sulfates but may be derivatized to include sulfates, or hydroxyl and amino groups and the like, for covalent linkage to peptides of the present invention. HA may also be modified with sulfates to alter the charge of the polymer.

Particle-free. The term “particle-free” as used herein to describe a polymer preparation (e.g., “particle-free hyaluronic acid” or “particle-free hyaluronic acid polymer analogue”), indicates that the polymer preparation, in the form to be administered according to the invention, is free of particulate forms of the polymer resulting from covalent cross-linking of two or more molecules of the polymer. In the present context “particle-free” has the same meaning as the term “non-crosslinked”. Polymer preparations used in the invention methods are at least predominantly particle-free, that is, the majority (>50%) of the mass of polymer in the preparation is not present in particles or cross-linked. Preferably, the polymer preparations are substantially (that is, at least about 70%) particle-free, and more preferably at least about 90% particle-free. Even more preferred are polymer preparations that are at least about 95%, 97% or 99% particle-free. Cross-linked polymer species in a composition of the invention may be detected and removed by molecular sieving chromatography or ultrafiltration methods known in the art. See, e.g., U.S. Pat. Nos. 5,166,331and 5,665,383.

Linker. A “linker” as defined herein is any molecule or covalent bond used to covalently link a peptide or other antigen to the hyaluronic acid polymer analogue. Many such linkers are well known in the art. For instance, on a polymer having carboxyl groups, such as hyaluronic acid, a carboxyl group of the polysaccharide may be linked to the N-terminus of a peptide chain through an acid amide linkage, where the single bond between the C and N in the amide linkage may be considered the linker. Such amide linkages may be formed using a condensation agent such as N,N′-dicyclohexylcarbodiimide, 1-ethyl-3-(3-dimthylaminopropyl)-carbodiimide hydrochloride or 1-ethoxycarbonyl-2-ethoxy-1,2-dihydroquinoline, as described, for instance, in U.S. Pat. No. 5,688,931 to Nogusa et al., which discloses a drug carrier comprising a polysaccharide derivative having a carboxyl group in which a peptide chain is introduced at a part or all of the carboxyl groups of the polysaccharide. The polysaccharide also can be conjugated to peptides for use in the present invention by use of bifunctional linkers, including some of the above compounds. Examples of bifunctional linkers which can be employed in the present invention to covalently link the MHC-recognized peptide and/or an epitope of an antigen associated with disease or a causative agent of disease include N-succinimidyl-3-(2-pyridyldthio)propinate (hereinafter “SPDP”) (Pharmacia, Piscataway, N.J.), which activates and allows formation of a bridge between two sulfhydryl groups of cysteines or a bridge between a derivatized (propinated-thiolyated) primary amino group and a cysteine; m-maleimidobenzoyl-N-hydroxy-succimide ester (hereinafter “MBS”) (Pierce Chemical, Rockford, Ill.), which activates an amino group and then couples by a sulfhydryl group to a cysteine sulfydryl so as to form a disulfide bond between the two polypeptides; and 1-ethyl-3-(3-dimethylaminopropyl)carbodiimide (hereinafter “EDC”) (Pierce Chemical, Rockford, Ill.), which can cross-link two polypeptides by sequentially activating the carboxyl group of one polypeptide and then adding such to an amino group of another polypeptide. N-isocyano-ethylmorphlin, bis-diazotized-benzidine, benzoquone and glutaraldehyde, which are other reagents commonly employed to link polypeptides, can be employed in the present invention and are available from Pierce Chemical, Rockford, Ill.; Eastman Kodak Chemicals, Rochester, N.Y.; Serva, Westbury, N.Y.; Sigma Chemical Co., St. Louis, Mo.; and E. Merck, Damstadt, West Germany (see Briand, J. S. et al, J. Immunol. Meth. 78:59 (1985); Kitagawa T. et al., J. Biochem. 79:233 (1976); Liu F. T. et al., Biochem. 18:690 (1979); Ternynck T. et al., Immunochem. 14:767 (1977); and Drevin, H. et al., J. Immunol. Meth. 77:9 (1985)).

Additionally, U.S. Pat. No. 5,856,299 to Righetto et al. discloses active esters of carboxy polysaccharides and semisynthetic derivatives of carboxy polysaccharides, wherein all or part of the carboxy groups thereof are esterified with an aromatic alcohol, a substituted aromatic alcohol, an aromatic heterocyclic alcohol, a substituted aromatic heterocyclic alcohol, an N-hydroxylamine, or a combination thereof. These active esters can be used for the preparation of modified carboxy polysaccharides or modified semisynthetic derivatives of such carboxy polysaccharides, in the form of esters, thioesters, or amides. Such active esters, modified polysaccharides, and modified semisynthetic derivatives of carboxy polysaccharides can be used for preparing conjugates of the present invention, preferably in soluble forms. Other carboxy polysaccharides and semisynthetic derivatives thereof employed in this patent are said to be described, for example, in U.S. Pat. Nos. 4,851,521, 5,122,598, 5,300,493, 5,332,809, and 5,336,668; European Patent Application No. 93917681.4; EP 0 216 453, EP 0 251 905, EP 0 342 557, EP 0 518 710, EP 0 603 264, and EP 0 605 478; and WO 93/06136 and WO 94/03499.

In addition, the Examples below illustrate several ways that a peptide or protein may be covalently coupled to an HA analogue, through a linker comprising a polyalkylene glycol or polyalkylene spacer group.

Epitope. The term “epitope” as used herein refers to a portion of an antigen, typically defined by a peptide, which is capable of eliciting a cellular or humoral immune response when presented in a physiologically relevant context in vivo. A “T cell epitope” refers to a peptide or portion thereof that binds to an MHC molecule and is recognized by T cells when presented in MHC molecules. A T cell epitope is capable of inducing a cell mediated immune response via direct or indirect presentation in heterodimeric membrane MHC molecules. Briefly, MHC molecules preferentially bind particular amino acid residues known as “anchor” residues (K. Falk et al., Nature 351:290-96 (1991)). This characterization permits Class I and II MHC recognition epitopes to be identified within any known peptide sequence. In the present context, the term “MHC recognition epitope” is synonymous with T cell epitope. “CTL epitope” refers to peptide sequences recognized by cytotoxic T lymphocytes (also called CD8⁺ cells) in association with Class I MHC. “Helper T cell epitope” refers to a peptide recognized by helper T cells (also called CD4⁺ cells). The term “B cell epitope” refers to a portion of an antigen, typically a peptide, capable of binding to an antigen binding site of an immunoglobulin and therefore capable of stimulating a humoral response without presentation in an MHC molecule.

Peptides useful in the present invention comprise at least one T cell epitope and, optionally, at least one B cell epitope; they also may include multiple T or B cell epitopes. When multiple epitopes are present in a peptide, the epitopes may be oriented in tandem or in a nested or overlapping configuration wherein at least one amino acid residue may be shared by two or more epitopes. Peptides may exceed eighteen amino acids when more than one T cell epitope is present or when additionally, a B cell epitope is present.

Peptide. The term “peptide” is used herein interchangeably with “oligopeptide” to designate a series of monomers or residues, typically L-amino acids, connected one to the other typically by peptide bonds between the alpha-amino and carbonyl groups of adjacent amino acids. The term peptide encompasses an isolated or recombinant sequence of amino acids, which may be naturally occurring or non-naturally occurring, and synthetic derivatives or analogues thereof. Sequences of naturally occurring amino acids recited herein utilize the standard amino acid nomenclature using single letter abbreviations for each residue—Alanine (A), Arginine (R), Asparagine (N), Aspartic acid (D), Cysteine (C), Glutamine (Q), Glutamic acid (E), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V). Amino acid “analogues” encompass functionally equivalent modified amino acid residues which are known in the art (see, e.g., U.S. Pat. Nos. 5,221,665 and 6,171,589). The term peptide as used herein also includes molecules completely devoid of amino acids which structurally and functionally mimic a peptide consisting of amino acids. For instance, U.S. Pat. No. 5,998,577 to Gevsen discloses a method of detecting or determining a sequence of monomer molecules which corresponds to an epitope on an antigen, or which has the equivalent molecular topology to that epitope. Such peptide mimetics of epitopes, which need not contain any amino acid, are known as “mimotopes”.

Formulations, Dosage and Administration

Conjugates of the invention may be formulated in pharmaceutical compositions for delivery via an appropriate route using formulations known in the art for other conjugates of HA polymer analogues, for instance, as described in various U.S. patents cited herein. Those skilled in the art will appreciate that the disclosed compositions of the present invention are preferably aqueous based preparations for administration to mammalians, and preferably humans. Thus, preferred aqueous preparations are sterile and substantially pyrogen-free with a pH of 6.0 to 7.8. The compositions typically comprise water in an amount of from about 60% to about 95% by based upon the total weight of the composition. The compositions may further comprise adjuvants known in the prior art, including aluminum salts and the MF59 microemulsion, and/or unconjugated peptides. Such adjuvants and unconjugated peptides are intended to amplify the immune response to the conjugate or generate a separate immune response.

Single or multiple administrations of the compositions can be carried out with dose levels and pattern being selected by the treating physician. Doses of any given peptide needed to obtain the desired immune response using the invention conjugates would typically be comparable to or less than described for an immunogenic dose of the same peptide formulated with a conventional adjuvant or carrier system. In general, a dose, as determined by the peptide concentration, will contain between 20 μg and 50 mg of peptide and the total peptide administered per day between 0.02 and 500 mg, however, the dose will vary with nature of the peptide and the species and size of the mammalian subject. Dosage regimen and frequency of administration will also vary depending on the intended function of the composition, for example inducing immunity (prevention) or a therapeutic for treating a recipient with the disease. In any event, the pharmaceutical formulations should provide a quantity of T and/or B cell stimulating epitopes covalently linked to a HA polymer analogue sufficient to effectively treat the recipient or prevent disease in the recipient. The compositions of the present invention may be administered by subcutaneous, transdermal, intradermal, intramuscular, intraocular, intrarectal, sub-buccal, oral, nasal, or intra-aural administration or by inhalation preparations.

Conjugates of the invention may be administered by any of the conventional routes of administration, including oral, intravenous, subcutaneous, intraperitoneal, intrathecal, intramuscular, intracranial, inhalational, topical, transdermal, suppository (rectal), pessary (vaginal), and the like.

Identification of Epitope Sequences within Peptides

Numerous disease-specific Class I and II MHC-presented (MHC-recognized) epitopes have been identified within protein antigen sequences and are available to the public. Such peptide antigen sequences that include Class I or Class II epitopes may derive from foreign proteins, cancers (e.g., overexpressed or mutated oncoproteins), or cryptic antigens that are designed to mimic disease-specific non-linear antigens recognized by the immune system.

Publications and large public databases are available for selecting a desired epitope. In addition, conserved motifs for Class I and II epitopes are known, permitting the identification of novel epitopes within known protein sequences. Algorithms are available to the public for screening peptide sequences and peptide databases to identify Class I and II epitopes in any known sequence. (Rammensee, Bachmann, Stevanovic, MHC Ligands and Peptide Motifs, Landes Bioscience, Georgetown, Tex. (1997); Rammensee, Friede Stevanovic: MHC ligands and peptide motifs: 1st listing, Immunogenetics 41, 178-228 (1995); Rammensee, “Cellular peptide composition governed by major histocompatibility complex class I molecules”, Nature 348:248-251 (1990); H. G. Rammensee, J. Bachmann, N. N. Emmerich, O. A. Bachor, S. Stevanovic: SYFPEITHI: database for MHC ligands and peptide motifs. Immunogenetics 50: 213-219 (1999; access via: http://www.uni-tuebingen.de/uni/kxi/); Thakallapally et al., “Motifscan”: A Web-based Tool to Find HLA Anchor Residues in Proteins or Peptides (http://hiv-web.lanl.gov/immunology/); Schreuder et al., The HLA dictionary 1999: Tissue Antigens 54:409-37(1999); Hiderhiro, A compilation of anchor residue motifs available at the Graduate School of Genetic Resources Technology, Kyushu University (http://www.grt.kyushu-u.acjp/˜hidehiro/public_old/motifs.html). In addition, a prediction algorithm for proteosomal cleavages can be used to identify cleavage sites that predict intracellular epitope formation via the proteosomal pathway for presentation of class I MHC ligand (see, e.g., NetChop at http://www.cbs.dtu.dk/services/NetChop/, or ProPrac at http://paproc.de/).

The following list of epitope sequences for presentation by Class I and II MHC and publications and references disclosing such epitope sequences is intended to exemplify peptide sequences for use in the present invention, but is not to be construed as limiting the scope of epitopes useful in the invention.

Epitope Sequences from Infectious Agents

An annually updated comprehensive list of HLA specified HIV epitopes is available from HIV Molecular Immunology Compendium (comprehensive listing of HIV-1 cytotoxic and helper T cell epitopes and antibody binding sites), Editors Korber, Brander, Havnes, Koup, Kuiken, Moore, Walker and Watkins. Los Alamos National Laboratory: Theoretical Biology and Biophysics, Los Alamos, N. Mex.; and De Groot et al., Brown University, “Interactive Web Site for MHC Ligand Prediction: Application to HIV Research AIDS Res Hum Retroviruses”, 13:7, 529-532 and web-based access via Epimatrix, http://tbhiv.biomed.brown.edu/. See also: Cosimi, L. A. and Rosenberg. E. S., Curr Opin Immunol Aug. 12, 2000 (4):375-80, “The Characterization of HIV-1 Specific CD4⁺ T Helper Epitopes”; Addo et al., PNAS, Feb. 13, 2001, Vol. 98, No. 4; 1781-1786, “HIV Tat and Rev epitopes determined by elispot”; Woodberry et al., J. Virol, July 1999, p. 5320-25, “Polytope Vaccine Containing multiple HLA A2 HIV CD8⁺ CTL Epitopes single artificial construct containing seven contiguous minimal HLA A2-restricted CD8⁺ CTL epitopes” (includes Nef, Pol, Gag and gp120 epitopes); Goulder et al., J. Virol June 2000; 74 (12), 5679-90, “African and caucasoid dominant Gag-specific epitopes presented by B42 and B81 ”; Atlfeld et al., J Virol September 2000;74(18):8641-9; HLA B60 and B61 restricted CTL epitopes (higher prevalence in Asian populations): Nef epitope; Atfeld et al., J Virol February 2000;75(3):1301-11, “HLA-A2 superfamily restricted HIV-1 CTL epitopes for prophylactic and therapeutic HIV-1 vaccines”; Goulder et al., J. Virol February 2000; 75(3):1339-47, “HLA-A*3002 (prominent in African populations) restricted CTL epitopes in p17-Gag”; Belyakov et al., PNAS Feb. 17, 1995;95 17;95(4):1709-14, “Mucosal CTL to HIV-1 gp160”; U.S. Pat. No. 5,562,905,

HIV Helper-T Cell Epitopes:

p17(21-35)—LRPGGKKKYKLKHIV; p17(22-29)—RPGGKKYX; p17(93-107)—EIKDTKEALDKIEEE; p17(118-132)—AAADTGHSSQVSQNY; p24(1-15)—PIVQNIQGQMVHQAI; p24(11-30)—VHQAISPRTLNAWVKVVEEK; p24(31-46)—AFSPEVIPMFSALSEC; p24(51-82)—DLNTMLNTVGGHQAAMQMLKETINEEAAEWDR; p24(96-103)—MREPRGSD; p24(111-132)—LQEQIGWMTNNPPIPVGEIYKR; p24(131-145)—KRWIILGLNKIVRMY; p24(156-174)—QPKEPFRDYVDRFYKTLRA; p2p7p1p6(55-69)—KEGHQMKDCTERQAN; p2p7p1p6(76-83)—PSYKGRPG; p2p7p1p6(98-112)—ESFRSGVETTTPPQK; RT(38-52)—CTEMEKEGKISKIGP; RT(88-102)—WEVQLGIPHPAGLKK; RT(251-261)—SSTVNDIQKLV; RT(285-299)—GTKALTEVIPLTEEA; RT(553-560)—SAGIRKVLFLD; Integrase(215-227)—KQITKIQNFRVYY; Vif(65-76)—VITTYWGLHTGE; Vif(81-96)—LGQGVSIEWRKQRYST; Vpr(66-80)—QLLFIHFRIGCRHSR; Tat(16-35)—SQPKTACTTCYCKKCCFHCQ; Tat(46-65)—SYGRKKRRQRRRPPQGSQTH; Tat(67-86); VSLSKQPTSQPRGDPTGPKE; Rev(16-35)—VRLIKFLYQSNPPPNPEGTR; Rev(25-39)—SNPPPNPEGTRQARR; Rev(76-95)—PPLERLTLDCNEDCGTSGTQ; gp160(38-48)—VYYGVPVWKEA; gp160(74-85)—CVPTNPVPQEVV; gp160(102-116)—EQMHEDIISLWDQSL; gp160(105-117)—HEDIISLWDQSLK; gp160(112-141)—WDQSLKPCVKLTPLCVSLKCTDLGNATNTN; gp160(155-169)—KNCSFNISTSIRGKV; gp160(220-234)—PAGFAILKCNNKTFN; gp160(223-231)—FAILKCNNK; gp160(235-247)—GTGPCTNVSTVQC; gp160(280-296)—NAKTIIVQLNESVAIC; gp160(308-322)—RIQRGPGRAFVTIGK; gp160(309-325)—IQRGPGRAFVTIGKIGN; gp160(321-336)—RIIGDIRKAHCNISRY; Nef(31-50)—GAASRDLEKHGAITSSNTAA; Nef(61-80)—QEEEEVGFPVTPQVPLRPMT; Nef(91-110)—LKEKGGLEGLIHSQRRQDIL;Nef(166-185)—HPVSLHGMDDPEREVLEWRF; Nef(182-205)—EWRFDSRLAFHHVARELHPEYFKN.

HIV CTL-Epitopes:

p24(8-20)—GQMVHQAISPRTL; p24(8-27)—GQMVHQAISPRTLNA-WVKVV; p24(8-32)—VHQAISPRTLNAWVK-VVEEKAF; p24(12-20)—HQAISPRTL; p24(13-23)—QAISPRTLNAW; p24(15-23)—LSPRTLNAW, DTVLEDINL, SLYNVATL, LSPRTLNAW, YPLTFGWCF; p24(16-24)—SPRTLNAWV; p24(21-40)—NAWVKVVEEKAFSPE-VIPMF; p24(28-47)—EEKAFSPEVIPMFSALS-EGA; p24-(35-43)—EVIPMFSAL; p24(36-43)—VIPMFSAL; p24(47-56)—ATPQDLNMML; p24(48-56)—TPQDLNTML; p24(51-59)—DLNTMLNTV; p24(51-70)—DLNTMLNTVGGHQAA-MQMLK; p24(61-69)—GHQAAMQML; p24(65-73)—AMQMLKETI; p24(83-92)—VHPVHAGPIA; p24(101-120)—GSDIAGTTSTLQEQIG-WMTN; p24(108-118)—TSTLQEQIGWF; p24(121-140)—NPPIPGEIKRWIILGNIK; p24(122-130)—PPIPVGDIY; p24(127-135)—GEIYKRWII; p24(131-139)—KRWIILGLN; p24(131-146)—KRWIILGLNKIVRMYC; p24(132-145)—KWILGLNKIVRMY; p24(137-145)—GLNKIVRMY; P24(166-174)—DRFYKLTRA; p24(174-184)—AEQASQEVKNW; p24(193-201)—NANPDCKTI; p24(195-202)—NPDCKTIL; p2p7p1p6(5-13)—SQVTNPANI; p2p7p1p6(55-70)—KEGHQMKDCTERQAN-F; p2p7p1p6(83-97)—GNFLQSREPEPTAPPF; p2p7p1p6(121-130)—YPLTSLRSLF; gp160(2-10)—RVKEKYQHL; gp160(29-49)—AAEQLWVTVYYGVPV-WKEAT; gp160(33-42)—KLWVTVYYGV; gp160(37-46)—TVYYGVPVWK; gp160(62-80)—DTEVHNVWATHACVP-TDPN; gp160(78-86)—DPNPQEVVL; gp160(105-117)—HEDIISLWDQSLK; gp160(121-129)—KLTPLCVTL; gp160(192-200)—KLTSCNTSV; gp160(208-217)—VSFEPIPIHY; gp160(212-231)—PIPIHYCAPAGFAILKC-NNK; gp160(297-322)—TRPNNNTRKRIRIQRG-PGRAFVTIGK; Nef(13-20)—WPTVRERM; Nef(62-81)—EEEEVGFPVTPQVPLRPMTY; Nef(66-97)-Nef(66-97 LAI)—VGFPVTPQVPLRPMTYK-AAVDLSHFLKEKGGL; Nef(68-76)—FPVTPQVPL; Nef(71-81)—RPQVPLRPMTY; Nef(72-79) VPLRPMTY; Nef(73-82)—QVPLRPMTYK; Nef(74-81)—VPLRPMTY; Nef(82-101)—KAAVDLSHFLKEKGG-LEGLI; Nef(86-94)—DLSHFLKEK.

HIV B Cell Epitopes:

P17(11-25)—ELDKWEKIRLRPGGKTLY; p17(12-29)—ELDKWEKIRLRPGGKTLY; p17(17-22)—IRLPGGKKYMLKHVVWAA; p17(30-52)—KLKHIIWASRELERFAVNPGLLE; p17(51-65)—LETSEGCRQILGQLQ; p17(86-115)—YCVHQRIEIKDTKEALDKIEEEQNKSKKKA; p17(86-115)—YSVHQRIDVKDTKEALEKIEEEQNKSKKKA; p17(113-122)—KKAQQAAADT; p17(112-122)—KKAQQAAADT; p17(119-132)—AAGTGNSSQVSQNY; p17(121-132)—DTGHSSQVSQNY; p24(1-20)—PIVQNIQGQMVHQAISPRTL; p24(11-25)—VHQAISPRTLNAWVK; p24(45-50) -EGATPQ; p24(46-56)—GATPQDLNTML; p24(51-61)—DLNTMLNTVG; p24(71-81)—ETINEEAAEWD; p2p7p1p6(1-5)—LAEAMS; p2p7p1p6(19-28)—NFRNQRKIVK; p2p7p1p6(45-54)—PRKKGCWKCG; p2p7p1p6(66-81)—RQANFLGKIWPSYKGR; p2p7p1p6(78-86)—YKGRPGNFL; Gag p17(12-19+100−105 IIIB)—ELDRWEKI+ALDKIE; P24(121-240 IIIB); p24(dis BRU)—DIRQGP+QGVGGP; Pro(1-7)—PQIYLWQ; Pro(36-46)—MSLPGRWKPKM; Pro(38-45)—LPGRWKPK; RT(294-302)—PLTEEAELE; RT(294-318)—PLTEAELELAENREILKEPVHGVY; RT(295-304)—LTEEAELELA; RT(442-450)—VDGAANRET; RT(536-549)—VPAHKGIGGNEQVD; Vif(34-46)—KARGWFYRHHYESP; Vif(176-192)—KPQKTKGHRGSHTMNGHX; Tat(2-15) -EPVDPNLEPWNHPS; Tat(2-17); EPVDPRLEWKHPGSQ; Tat(73-86)—PTSQPRGDPTGPKE; Rev(32-50)—EGTRQARRNRRRWRERQR; Rev(70-84)—PVPLQLPPLERLTLD; Rev(70-84)—PVPLQLPPLERLTLD; Rev(96-105)—GVGSPQILVE; gp160(30-51)—ATEKLWVTVYYGVPVWKEATTT; gp 160(41-50)—GVPVWKEATT; gp160(51-70)—LFCASDAKAYDTEVHNVWAT; gp160(61-70)—YDTEVHNVWA; gp160(73-92)—ACVPTDPNPQEVVLVNVTEN; gp160(81-90)—PQEVVLVNVT; gp160(81-100) -PQEVVLVNVTENFDMWKNDM; gp160(83-92)—EVVLVNVTEN; gp160(308-322)—RIHIGPGRAFYTTKN; gp160(309-315)—IHIGPGR; Env(dis)-gp120(V3 309-318+329-338)—IQRGPGRAFV+AHCNISRAKW; Nef(11-20)—VGWPTVRERM; Nef(15-24)—TVRERMRRAE; Nef(30-43)—VGAASRDLEKHGAI; Nef(31-40)—GAASRDLEKH; Nef(31-50)—GAASRDLEKHGAITSSNTAA; Nef(60-73)—AQEEEEVGFPVTPQ; Nef(83-88)—AAVDLS; Nef(83-103)—AAVDLSHFLKEKGGLEGLISH; Nef(148-157)—VEPDKVEEAN; Nef(151-170)—DKVEEANKGENTSLLHPVSL; Nef(158-181)—KGENTSLLHPVSLHGMDDPEREVL; Nef(171-190)—HGMDDPEREVLEWRFDSRLA

Other Viral, Bacterial and Parasite Derived Epitopes:

Herpes Simplex Virus Type 1 (HSV-1) is the causative agent of gingivostomatitis, herpes labialis, keratoconjunctivitis, and encephalitis. HSV-1 epitopes are derived from gB glycoprotein and VbetalOI/junctional sequence, among other antigens (Cose, S. C. and Carbone J. R., J. Virol. 69:5849-5852 (1995)).

Herpes Simplex Virus Type 2 (HSV-2) is the causative agent of genital herpes, neonatal herpes, aseptic meningitis, cervical carcinoma. HSV-2 epitopes are derived from gG2 (e.g., gG2(561-578)).

Cytomegalovirus causes congenital abnormalities in neonates, pneumonial, heterophile-negative mononucleosis, and retinitis. CMV protein antigens containing T cell epitopes are known (U.S. Pat. No. 6,251,399) and include epitopes within the viral capsid antigen (VCA), early antigens, nuclear antigen (EBN) and protein pp65.

Other viral and bacterial pathogen related epitopes are described in the related art. For example, measles (NP29 protein, especially residues (281-290)) (Hsu et al., Vaccine August 1996; 14(12):1159-66); and Foot-and Mouth Disease Virus (T and B cell epitopes are described, for example, in U.S. Pat. No. 6,107,021); B and T cell epitopes for Group A Streptococci (Pruksakom et al., J. Immun., 149:2729-2735, October 1992 and U.S. Pat. No. 6,174,528).

Tuberculosis derived epitopes include: MPT64 protein: (24-43)—APKTYCEELKGTDTGQACQI, (34-53)—GTDTGQACQIQMSDPAYNIN, (44-63)—QMSDPAYNINISLPSYYFDQ, (54-73)—ISLPSYYPDQKSLENYIAQT, (74-93) RDKFLSAATSSTFREAFYEL and others (Roche et al., Scand. J Immunol. 43, 662-670, 1996).

Cancer-Associated Epitopes:

Cancer associated peptides, which can be antigens not expressed by normal cells, such as viral antigens, or somatic genes which are overexpressed or expressed as mutated proteins. The following sequences and publications disclose illustrative epitopes for use in the present invention, but should not be construed at limiting.

Epitopes of melanoma antigens including MAGE-3, MAGE-3(161-169), (Marchard et al., Int. J. Cancer 1999, 80:219-230), and MART-1(27-35), (Wang et al., Clin. Cancer Res 1999, 5:2756-65), tyrosinase-related protein 2 (TRP2) epitopes: (125-133) VIRQNIHSL, (197-205) LLGPGRPYR, (180-188) SVYDFFVWL, (217-225) VTWHRYHLL, (288-296) SLDDYNHLV, (367-376) SLHNLVHSFL, (455-463) YAIDLPVSV, (387-395) ANDPIFVVL, (368-376) YMDGTMSQV, (485-493) GLFVLLAFL, (482-490) ALVGLFVLL, and Melan-A(27-35) AAGIGILTV (Sun et al., Int. J. Cancer: 87,399-404, 2000); and those identified by Robbins et al., Curr Opin. Immunol. 1996, 8:628-636.

Epitopes derived from colorectal carcinoma antigens, such as CEA, 17-1 A, B-72.3 and Ep-CAM. (Zhu et al., Clin. Cancer Res. 2000, 6:24-33).

Oncoprotein antigens such as p53 (Ropke et al., PNAS, 1996, 93:14704-07).

Epitopes of breast cancer antigens including HER2/neu (p 185) (Esserman et al., Cancer Immunol Immunother 1999, 47:337-42); IISAVVGIL, KIFGSLAF, PDTRPAPGSTAPPAHGVTSA; B cell epitope KASIFLK (Cao et al., Breast Cancer Res Treat 1999 Feb; 53(3):279-90; and ovarian cancer related B cell epitopes (Chinni et al., Cancer Immunol Immunother 1998 Mar; 46(1):48-54

Other tumor antigens including T cell epitopes include BAGE, CAGE-1, CAGE-R, MUM-1, CDK4 (Robbins et al., supra); AARAVFLAL, YRPRPRRY, VLPDVFIRC, AYGLDFYIL, SYLDSGIHF, EEKLIVVLF, ACDPHSGHFV, IISAVVGIL, KIFGSLAFL, YMLDLQPETT, PDTRPAPGSTAPPAHGVTSA

Viral Antigens Associated with Infections and Cancer:

Hepatitis B antigens: U.S. Pat. No. 5,932,224, e.g., HBpol(61-69)—GLYSSYVPV, HPpol(161-169)—GLYSSTVPV, HBpol(455-463),—GLSRYVARL, HBpol(773-382)—ILRGYSFVRV, HBpol(803-811)—SLYADSPSV, HBpol(816-824)—FLLSLGIHL; Vitiello et al., J. Clin. Invest. 1995 95:341-345; J. Immunol Oct. 15, 2000;165(8):4748-55; e.g., VLQAGFFLL, FLLTRILTI, FLGGTPVCL, LLCLIFLLV, LLDYQGMLP, WLSLLVPFV, GLSPTVWLS; HBsAg peptides S(179-186; FVQWFVGL) and S2(208-216; ILSPFLPLL), residues 122-137 (Dreesman et al., Adv. Exp. Med. Biol. 1985; 185:129-37).

Hepatitis C virus (HepC), J. Hepatol February 2000; 34(2):321-9) E2(614-622); immunodominant HLA-A24 epitope AYSQQTRGL; antigen NS5 (P17, residues 2423-2434), (Uno-Furuta et al., Vaccine Feb. 28, 2000; 19(15-16:2190-6).

Epstein-Barr virus (EBV) associated Hodgkin's lymphomas and nasopharyngeal carcinoma antigens: LMP1, LMP2, and EBNA-1, EBNA-3 (Orentas et al., Clin Immunol. 2001 Feb; 98(2):220-8 and Curr. Opin. Immunol. 1996, 8:651-657) and EBNA3B epitopes (399-408), (416-424), and latent cycle antigens including epitopes RRIYDLIEL, RRARSLSAERY, RRRWRRLTV, and FRKAQIQGL and CLG peptide (Marastoni et al., J. Med. Chem. 2001 Jul 5:44(14):2370-3); J. Immunol. 1995 154:5934-5943, E7 epitopes YLDLQPETT, LLMGTLGIV,.

Human Papilloma Virus (HPV): HPV-16 positive cervical cancer, HPV-6 and -11 positive anogenital warts and laryngeal papillomas, HPV-1 and -4 positive plantar warts. HPV epitope comprising antigens include E6 and E7 oncoproteins and recombinant “detoxified” E6/E7 fusion peptides (Tarpey et al., Vaccine February 1996;14(3):230-6).

### EXAMPLE 1

### HIV Vaccine Preparations for Inducing Cell Mediated Immune Response

Preparation of the peptide. p7g is a 9 amino acid peptide (m.w.=1064) and recognized CTL epitope (AMQMLKETI) derived from the p24 region of the HIV-1 Gag protein (Doe et al., AIDS. June 1996;10(7):793-4.). The p7g peptide was synthetically prepared using a commercially available peptide synthesizer and purified by reverse phase HPLC. The purified peptide was reconstituted in distilled water to a final concentration of 1 mg/ml and stored at −80° C.

Preparation of hyaluronic acid. Hyaluronic acid (HA) from human umbilical cord was obtained from Aldrich-Sigma for use in the present studies. The polymer size of these preparations was 6,000,000-8,000,000 Dalton. Alternative sources of HA include rooster comb, Streptococcus zooepidemicus and synthetic preparations. A smaller size (1,200,000 dalton) of HA is also commercially available from Lifecore Biomedical Inc. Smaller size HA can be made through enzymatic degradation of larger HA. Testicular hyaluronidase degrades HA to generate even-numbered oligosaccharides at different size based on different conditions (Mever K. in The Enzymes (P. D. Boyer, Ed) vol. 2, pp. 307-320, Academic Press, N.Y., 1971). By this method, even two unit HA fragments can be made (Pouyani, T., Bioconjugate Chem. 5:339-347 (1994)).

Preparation of covalently linked p7g-HA with an amide bond linkage. Peptide p7g was first linked to HA using the following scheme:

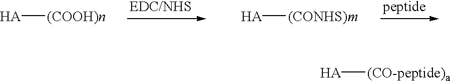

In this approach, one mg each of hyaluronic acid and N-hydroxysuccinimide was dissolved in 2 ml PBS (1:10) solution at room temperature. After 5.1 mg of 1-ethyl-3-(3-dimethylaminopropyl)-carbodiimide hydrochloride (EDC) was added, the mixture was adjusted to pH 5.0 with 0.1 N HCl solution and stirred at room temperature for 2 hours. To this solution, 1 mg of HIV peptide p7g, NH₂-AMQMLKETI-COOH, dissolved in 1 ml of distilled water was added and the mixture placed on a rocker and rocked gently at room temperature for 3 hours. The resulting mixture was dialyzed against 4 liters distilled water 3 times with MWCO=6000-8000 to remove free peptide and EDC. The distilled water was changed every 2 hours. The reaction was monitored with gel-filtration HPLC and C18 reverse phase HPLC. The resulting p7g-HA conjugate solution was lyophilized and stored at −80° C. The conjugate could be further purified by gel-filtration HPLC (Phenomenex Biosep S series).

Preparation of covalently linked p7g-HA through polyalkyleneamine and polyalkylene glycol amine spacer using ethylenediamine as an example. This approach uses the following scheme:

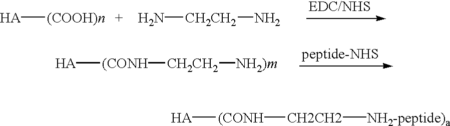

Although ethylenediamine is exemplified here, the polyalkyleneamine can be spermidine, spermine, Jeffamine and other polyamines that could carry multiple peptides per-linker.

Five mg hyaluronic acid and 26 mg 1-ethyl-3-(3-dimethylaminopropyl)-carbodiimide hydrochloride (EDC) were dissolved in 5 ml PBS (1:10) solution at room temperature. After a solution of 8.0 mg ethylenediamine in 0.4 ml distilled water was added, the mixture was adjusted to pH 5.0 with 6 N HCl solution, and stirred at room temperature for 4 hours. The resulting mixture was dialyzed against 4 liters of distilled water 3 times. The distilled water was changed every 2 hours. The solution was lyophilized. White solid was obtained. This white solid was mixed with NHS-activated peptide at room temperature. The mixture was put on a rocker for 5 hours before being dialyzed with MWCO 12K to 14K. The resulting solution was lyophilized to obtain the final HA-conjugated peptide. The crude product may also be purified with gel-filtration chromatography.

Preparation of covalently linked HA-O-CO-protein/peptide via ester linkage. This approach uses the following scheme:

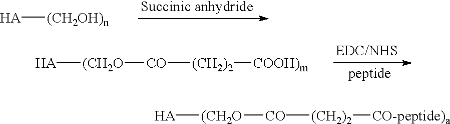

For this approach, dissolve HA in a PBS (1:10) buffer having pH at 7.3. Add 10 molar excess of succinic anhydride in PBS. React overnight at room temperature. Remove excess reactants from the succinylated HA by dialysis or gel filtration. The resulting solution is then lyophilized. The succinylated HA is redissolved in 0.1 N MES buffer, pH 4.75. A 20 molar excess of 1-ethyl-3-(3-dimethylaminopropyl)-carbodiimide hydrochloride (EDC) and 5 molar excess of N-hydroxylsuccimide is added while maintaining pH 5. The reaction mixture is stirred at room temperature for 4 hours. A solution of desired peptide/protein solution is added in. The mixture is then stirred at room temperature overnight. Excess reactants are removed from the reaction mixture by dialysis or gel filtration. The product is obtained after lyophalization.

Preparation of covalently linked HA-protein/peptide/oligonucleotide via thio-linkage. This approach uses the following scheme:

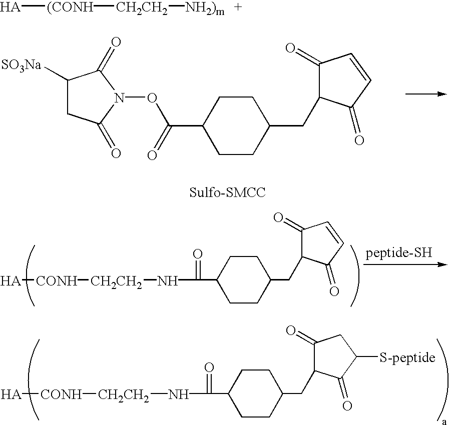

For this approach, dissolve 10 mg of aminonated HA in 2 ml of 0.1 M sodium phosphate, 0.15 M NaCl, pH 7.2. Add 10 molar excess of sulfo-SMCC in the above solution. Mix gently to dissolve. The mixture is put on rocker and rotated gently for 2 hours at room temperature. Immediately purify the resulting maleimide-activated HA by applying the reaction mixture to a desalting column of Sephadex G-25. Mix the maleimide-activated HA at the desired molar ratio with peptide/protein/oligonucleotide dissolved in 0.1 M sodium phosphate, 0.15 M NaCl, pH 7.2, and incubate overnight. The resulting mixture is purified by dialysis or gel filtration. Final product is obtained by lyophilization.

Single Dose Vaccination Protocol. Six to eight week old female BALB/c mice (purchased from Harlan Laboratories, Frederick, Md.) were divided into six groups of five mice each. Mice were immunized with one of three preparations: a control or one of two peptide vaccine preparations. 150 μl of sterile saline was used for control mice. The vaccines were of equivalent volume and contained either 30 μg of free p7g peptide, or p7g peptide covalently linked to HA (30 μg peptide per dose). Three modes of administration were compared: intramuscular (i.m.), subcutaneous (s.c.), and intrarectal (i.r.). Subcutaneous injections were delivered at the base of the tail and i.m. injections delivered as a divided dose to each quadricep. Intrarectal injections were administered into the rectal submucosa. All animals used in this study were maintained at Vaxim, Inc., Rockville, Md., under the supervision of Maryland Laboratory Animal Resources.

In vivo Immune Challenge with HIV-1 Gag Recombinant Vaccinia Virus. Thirty days after the single vaccination, mice were challenged by intraperitoneal administration of recombinant vaccinia virus (10⁷ PFU/mouse) containing the HIV-1 Gag gene (vP1287, catalog no. 3542; NIH AIDS Research and Reference Reagent Program) in accordance with methods known in the art (Belyakov, I. M. et al., “Mucosal immunization with HIV-1 peptide vaccine induces mucosal and systemic cytotoxic T lymphocytes and protective immunity in mice against intrarectal recombinant HIV-vaccinia challenge”, Proc Nat'l Acad Sci USA 95:1709-1714 (1998); Qiu et al., J. Virol. 2000, 74:5997-6005).

Splenocytes were harvested 5 days after recombinant vaccinia virus challenge (Oiu et al., J. Virol. 1999, 73:9145-152). Briefly, spleens were removed and compressed through sterile nylon mesh with a rubber stopper, then washed twice with RPMI 1640. The harvested cells were centrifuged for 10 minutes in a Sorval H-1000B rotor at 2,000 rpm (200×g) and resuspended for the ELISPOT and ICC assays described below. Cell viability was determined by trypan blue exclusion.

The cytotoxic lymphocyte (CTL) response was assessed by intracellular staining and flow cytometric analysis and the specificity of the CTL response determined by ELISPOT assay.

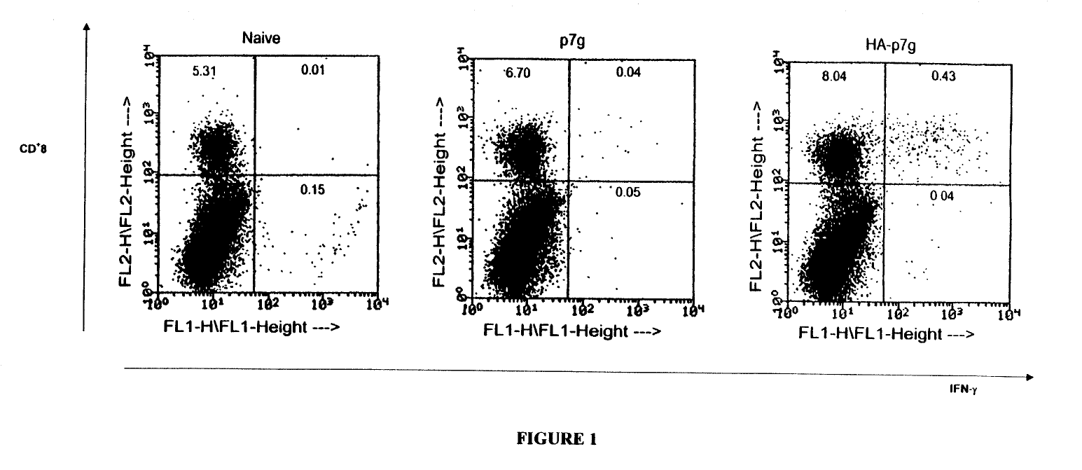

Quantification of CTL Response by Intracellular Staining and Flow Cytometry. Splenocytes obtained 5 days post-viral challenge were isolated from mice vaccinated with either free peptide, peptide-HA conjugate, or control saline. Freshly harvested cells or cells frozen in liquid nitrogen may be used. Frozen cells were cultured in vitro with or without p24 peptide for 24 hours. The p24 peptide was added at a concentration of 2 μg/ml for 24 hours, then Golgistop (Pharmingen, San Diego, Calif.) was added 6 hours before the cells were harvested. The cells were then washed once in fluorescence-activated cell sorter buffer and stained with phycoerythrin-conjugated monoclonal rat anti-mouse CD8 and PerCP-CY5.5-conjugated rat anti-mouse CD4 antibody (Pharmingen). Intracellular cytokine staining was carried out as suggested by the manufacturer (Pharmingen) using the Cytofix/Cytoperm kit. Fluorescein isothiocyanate-conjugated INF-γ antibodies and the imnmunoglobulin isotype control antibody (rat IgG1) were all purchased from Pharmingen. Analysis was performed on a Becton Dickinson FACScan with Lysis II Software (Becton Dickinson Immunocytometry Systems, Mountain View, Calif.) or Coulter Epics XL/XL-MCL Flow Cytometry System (Fullerton, Calif.). The results are graphically depicted in FIG. 1.

### Results

Negative control mice immunized with saline demonstrated the expected distribution of CD4⁺ and CD8⁺ cell populations with no evidence of INF-γ expression. (FIG. 1). Mice immunized with free p7g peptide showed a similar CD4/CD8 profile with no CTL or INF-γ expression. (FIG. 1) In contrast, the mice immunized with p7g covalently linked to HA demonstrated clonal expansion of CD4⁺ cells and a marked CTL response with CD8⁺ proliferation and INF-γ expression by CD8⁺ but not CD4⁺ cells.

ELISpot ASSAY. The ELISpot assay was performed as described by Miyahira et al. (J. Immunol. Method. 181:45-54 (1995)) and Murali-Krishna et al. (Immunity 8:177-187 (1998)), modified to detect HIV-1 p7g-specific CD8⁺ T cells. Splenocytes from immunized, vaccinia challenged mice were harvested as described above, and cultured in vitro with or without p24 peptide for 24 hours (sequence derived from HIV-1 Gag protein). Briefly, ninety-six-well filtration plates (Millipore, Bedford, Mass.) were coated overnight at 4° C. with 50 μl (10 μg/ml) of anti-mouse IFN-γ (R46A2; Pharmingen) in sterile PBS. The plates were blocked for 2 h at 37° C. with sterile RPMI 1640 containing 10% fetal calf serum and 1% bovine serum albumin and were washed three times with sterile PBS. Various dilutions of splenocytes in 200 μl of complete medium with or without MHC class I-restricted p24 peptide (aa AMQMLKETI) were placed in each well and incubated at 37° C. for 24 h. Plates were washed with PBS containing 0.025% Tween-20 and were overlaid with 50 μl (5 μg/ml) of biotinylated anti-mouse IFN-γ (XMG1.2; Pharmingen). The plates were washed six times with PBS containing 0.025% Tween-20 and were treated with 1.25 μg of avidin-conjugated alkaline phosphatase (Sigma) per ml for 2 h at room temperature. After a final wash with PBS, IFN-γ spot-forming cells were detected by the addition of BCIP-nitroblue tetrazolium solution (Sigma) and were counted with a stereomicroscope. The reported values reflect the mean of duplicate samples recorded in spot forming units (SFU) per million cells.

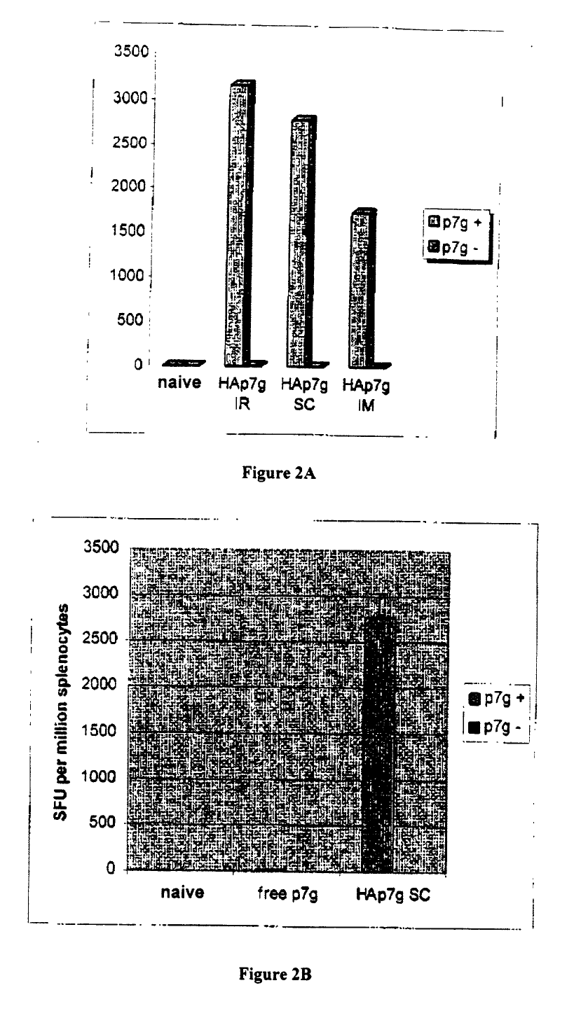

ELISpot Assay Results. p7g-specific CD8⁺ cells obtained from mice vaccinated with the p7g-HA conjugate did not express IFN-γ in the absence of p24 peptide stimulation, whereas when cells from the same animals were cultured in the presence of p24 peptide, IFN-γ secreting p7g-specific CD8⁺ cells were detected and ranged from 500 to 3200 SFU per million cells (FIG. 2A). This result represents a 10-50 times greater antigen specific CTL response than observed with free peptide (FIG. 2B).

Intramuscular and intrarectal administration elicited a greater CTL response than subcutaneous administration, however the s.c. route elicited a CTL response that was ten times greater than that observed with other vaccines.

The control mice vaccinated with saline were negative for IFN-γ when cultured with or without p24 peptide.

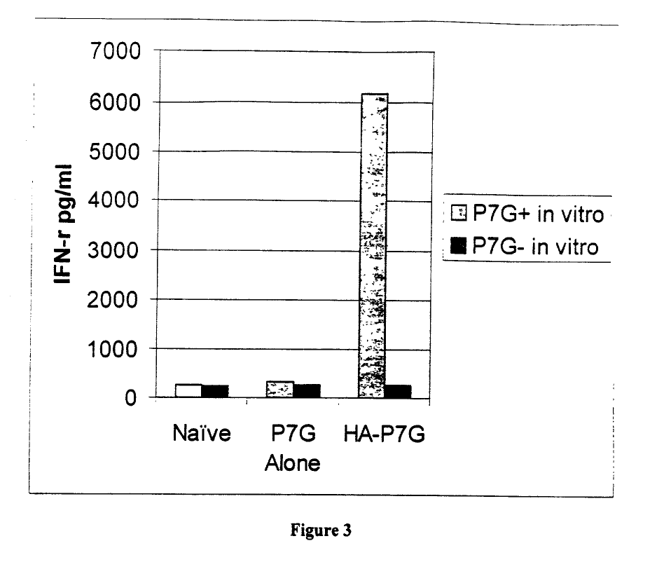

In vitro cytokine assay. Mouse spleen cells were cultured with p7g peptides in parallel with the ELISpot assays. Culture supernatants were collected 24 hours after addition of peptide, and the concentrations of gamma interferon (IFN-γ) were determined by enzyme-linked immunosorbent assay (ELISA). Briefly, anti-mouse IFN-γ (R46A2; monoclonal antibody, Pharmingen) was diluted in coating buffer (pH 9.6) and coated (1 μg protein/well) onto 96-well enzyme-linked immunosorbent assay (ELISA) plates (Nunc Immunoplates; Nunc. Corp., Naperville, Ill.) by overnight incubation at 4° C. The plates were washed six times in wash buffer, then blocked with PBS containing 0.05% Tween 20 (Sigma), 1% low-fat dry milk (Carnation; Nestle Food Corp., Glendale, Calif.), and 4% goat serum (KPL Laboratories, Gaithersburg, Md.) for 1 hour. After washing, each plate was incubated with samples diluted in PBS containing 1% BSA and 0.05% Tween 20 for 1 hour. The plates were then incubated with rabbit anti-mouse IFN-γ (Cytlmmune Sciences, Inc. College, Md.) diluted 1:1000 in the same PBS solution for 1 hour and washed again six times. The goat anti-rabbit IgG alkaline phosphatase conjugate was then added to each plate and incubated for 30 min. The bound mouse IFN-γ was quantitated by calorimetric reaction using p-nitrophenyl phosphate as a substrate (KPL Laboratories). The optical density (OD) was measured at 405 nm in a Bio-Tek Plate Reader (Winooski, Vt.). All samples and 110 dilutions were tested in duplicate. For these assays, the limit of detection was 5 pg of IFN-γ per ml. The results shown in FIG. 3 demonstrate that free peptide p7g induced no measurable IFN-γ whereas p7g coupled to HA induced about 6000 pg/ml IFN-γ.

### EXAMPLE 2

### Induction of Antibody with a Peptide-HA Conjugate without Adjuvant

Peptide T, which is derived from the V2 region of HIV-1, inhibits replication of R5 and dual-tropic (R5/X4) HIV-1 strains in monocyte-derived macrophages (MDMs), microglia, and primary CD4(+)T cells. Picone, D. et al., “Peptide T revisited: conformational mimicry of epitopes of anti-HIV proteins”, J Pept Sci 7:197-207 (2001). Peptide T (AS TTTNYT) corresponds to residues 185-192 of gp120, the coat protein of HIV and is endowed with several biological properties in vitro, notably inhibition of the binding of both isolated gp120 and HIV-1 to the CD4 receptor, and chemotactic activity. The effect on the humoral response to peptide T of conjugation to HA was examined.

Female BALB/c mice (6 weeks of age) received two vaccinations containing peptide T that was either mixed with Freund's adjuvant (complete and incomplete) or covalently linked to HA in accordance with the present invention using the methods described above (Example 1). Mice were primed with 100 μg antigen on day 0 and boosted with the same amount on day 14. Sera were collected and ELISA were done according the methods described above. Naive mice served as negative controls.

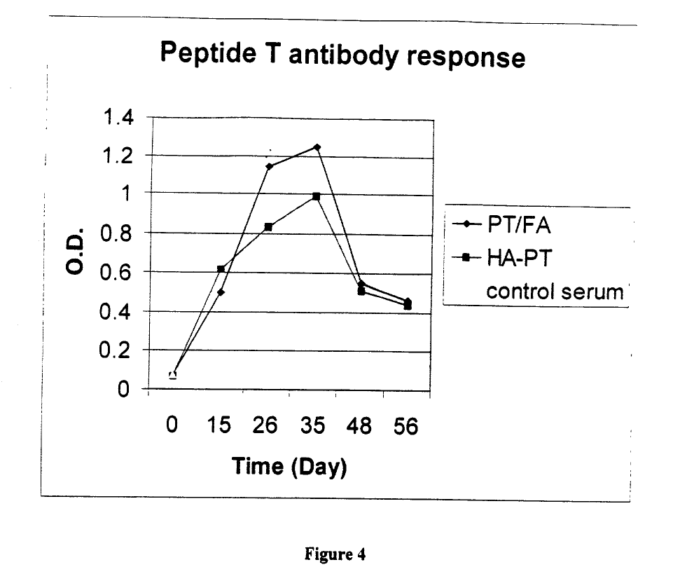

IgG titers were compared 56 days following the second vaccination with peptide T vaccine formulation. Results were obtained in 1:100 dilutions. As shown in FIG. 4, the antibody titer to peptide T in mice vaccinated with the peptide linked to HA was comparable to control mice with peptide T in Freund's adjuvant. This was an entirely unexpected result, as it is well known in the art that humoral responses to peptides are poor in the absence of adjuvants.

It is to be understood that the above detailed examples are intended to illustrate and not limit the scope of the invention. Other aspects, advantages and modifications within the scope of the invention will be apparent to those skilled in the art to which the invention pertains.

What is claimed is:

1. A method of modulating an immune system response to an antigen in a mammal, said method comprising administering to said mammal a particle-free therapeutic comprising a hyaluronic acid polymer analogue covalently linked to at least one peptide that comprises a T cell epitope recognized by an MHC molecule of said mammal, wherein said T cell epitope is defined by a sequence of at least about eight amino acids of said antigen.

2. The method of claim 1 wherein said T cell epitope is recognized by an MHC Class I molecule and by a CD8⁺ T cell of said mammal.

3. The method of claim 1 wherein said T cell epitope is recognized by an MHC Class II molecule and by a CD4⁺ T cell of said mammal.

4. The method of claim 2 wherein said immune system response comprises a cytotoxic T lymphocyte that recognizes said antigen.

5. The method of claim 2 wherein said immune system response comprises a CD4⁺ T cell that recognizes said antigen.

6. The method of claim 1 wherein said immune system response comprises an antibody that recognizes said antigen.

7. The method of claim 1 wherein said immune system response to said antigen is increased after administration of said conjugate.

8. The method of claim 7 wherein said antigen is an antigen of a pathogenic agent or a tumor cell.

9. The method of claim 3 wherein said immune system response to said antigen is decreased after administration of said conjugate.

10. The method of claim 9 wherein said antigen is an antigen of a tissue or organ transplanted to said mammal.

11. The method of claim 1 wherein said step of administering further comprises administering a plurality of conjugates each comprising at least one peptide that comprises a T cell epitope recognizable by an MCH molecule linked to a hyaluronic acid polymer analogue wherein the peptides of at least two such conjugates are different.

12. A method of improving MHC presentation of a T cell epitope of an antigen in a mammal comprising administering to said mammal a conjugate comprising particle-free hyaluronic acid polymer analogue covalently linked to a peptide that comprises a T cell epitope recognized by an MHC molecule of said mammal, wherein said T cell epitope is defined by a sequence of at least about eight amino acids of said antigen.

13. A pharmaceutical composition for administration to a mammal for improving MHC presentation of a T cell epitope of an antigen in said mammal, said composition comprising a conjugate comprising particle-free hyaluronic acid polymer analogue covalently linked to a peptide that comprises a T cell epitope recognized by an MHC molecule of said mammal, wherein said T cell epitope is defined by a sequence of at least about eight amino acids of said antigen.

14. A composition of matter comprising particle-free hyaluronic acid polymer analogue covalently linked to at least one peptide comprising a T cell epitope recognized by an MHC molecule of a mammal.
